(19) United States
(12) Patent Application Publication
(10) Pub. No.: US 2021/0366591 A1
(43) Pub. Date: Nov. 25, 2021

(54) Client mental health assessment and treatment systems using isochronic sounds, tones and/or waves focusing on therapy addressing the root causes and triggers of negative emotional states or conditions

(71) Applicant: Sardar Nadeem Khan, dunnellon, FL (US)

(72) Inventor: Sardar Nadeem Khan, dunnellon, FL (US)

(21) Appl. No.: 16/882,658

(22) Filed: May 25, 2020

(51) Int. Cl. G16H 20/70; G16H 20/30; G16H 20/60; G16H 80/00; G16H 10/20; G16H 40/63; G16H 40/67

(52) U.S. Cl. CPC G16H 20/70; G16H 20/30; G16H 20/60; G16H 40/67; G16H 10/20; G16H 40/63; G16H 80/00

(57) ABSTRACT

A platform and system to help clients get mental health care services provided by their businesses and clinics 24 hours from anywhere and help clients get assessment and treatment systems addressing the root causes and triggers of negative emotional states or conditions

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

One or more embodiments of the invention generally relate to client mental health assessment and treatment systems using isochronic sounds, tones and/or waves. More particularly, certain embodiments of the invention relate to systems focusing on therapy addressing the root causes and triggers of negative emotional states or conditions rather than solely medicating the client.

### Description of the Related Art

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. In a traditional way, hospitals and other medical or health-related service providers may seek to alleviate client discomfort by prescribing medication resulting in the immediate but temporary suppression of physical symptoms rather than comprehensively addressing the root causes of an underlying ailment. However, in a controlled substance addiction or mental health (e.g., chronic anxiety) context, therapy can also include psychological counseling with a professional in addition to medicine, potentially further complicating client treatment and prolonging recovery times. Nevertheless, “it has been estimated that 75-90 percent of all visits to primary care physicians are for stress related problems.” [Source: https://www.stress.org/americas-1-health-problem; Retrieved on May 30, 2019; Job Stress: America's Leading Adult Health Problem, by Paul J. Rosch, M.D., F.A.C.P., in USA Magazine, May 1991; American Academy of Family Physicians Survey, 1988, U.S. News & World Report, Dec. 11, 1995; also, Research by Perkins (1994) cited in the Harvard Business Review showed that 60% to 90% of doctor visits were stress-related.] Such visits may include, but are not limited to, numerous feelings and belief systems related causes such as: depression, fears, anger, sadness, addiction, and phobias.

Hospitals, clinics, alcohol and drug addiction rehabilitation centers may not have dedicated automatic in-house (e.g., inclient and outclient) capabilities to remotely provide their clients with the necessary supplemental treatment to assist in the reduction of stress and the various emotional triggers associated with the production of stress. Such emotional triggers can contribute to the onset of various diseases and illnesses, and can include symptoms such as addiction. Identifying and promptly counteracting these emotional triggers can be critical in helping clients recover and in providing better overall care. Thus, establishing in-house education and treatment programs by caretakers, e.g., inclusive of both licensed and non-licensed (but certified) professionals, can offer a cooperative solution regarding helping clients cope with their stress, anxiety, or other negative mood state, by providing an accessible and creative outlet geared towards addressing and relieving such mood states. Such an automated solution permits mental health clients to not only benefit by living a healthier lifestyle through careful and ongoing remote monitoring by qualified persons, but also positions clients to better handle the primary medical treatment they are concurrently receiving.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. Computer program products and methods of use thereof for mental health treatment can include performing a sequence of operations on a client, including guiding the client to an area of the mind needing treatment, which may be then softened to facilitate acceptance of the disorder. Emotional traumas are isolated and may be computationally resolved to form resolutions, which are prevented from reactivation. Next, positive reinforcement history of maladaptive coping behaviors is treated and protected resulting, as a practical application of the disclosed operations the neutralization of the maladaptive emotions of the client.

Particulars include effective energy psychology application such as through Thought Field Therapy (“TFT”), Emotional Freedom Technique(s) (“EFT”), and/or Tapas Acupressure Technique (“TAT”), which may be applied once the program product has accessed a targeted history of the client.

By way of educational background, another aspect of the prior art generally useful to be aware of are methods of training resulting in experienced individuals enhanced intimacy related to an improved sensual and sexual experience. Computers connected to servers via various internet connections or wireless connections can be used by individuals to experience enhanced intimacy. Simulation and testing are offered through interactivity or game-play, either or both of which may engage the trainee in using what was taught and also tests the trainee's level of understanding. The methods may include brainwave entrainment audio/music to enhance lovemaking, Natural Language Processing (“NLP”, referring to a subfield of computer science concerned with the interactions between computers and human languages), and Color Treatment (referring to choosing the most universal meanings applied to color and uses them to assist in helping clients to control their emotions and emotional responses).

Also, methods are known for facilitating a participant to reduce or eliminate anxiety over-reactions by playing of meditative background music. A program is used to collect, assess and store responses from inquiry into said participant's history of significant events. The participant, via the program, is given a set of directions and presented to with a significant temperature change to said participant's body or a portion thereof for a period of time while using said directions and a means for recording data from sensors or from said participant. Methods to play the client's desired meditative background music through, for example, a CD player, MP3, WAV or other digital medium for sending through a wired or wireless connection are provided.

Known techniques and mental illness treatment methods have included investigations into auditory beat stimulation, which may be a promising new tool for the handling of cognitive processes and the variation of mood states. Auditory steady-state responses may have been shown to have a relationship to auditory beat stimulation (ABS) and otherwise impact the design of appropriate stimulation protocols. Monaural- and binaural-beat frequencies have been shown to play a role in cognition and mood states, in addition to their efficacy in targeting disease symptoms. We aim to highlight important points concerning stimulation parameters and try to address why there are often contradictory findings with regard to the outcomes of ABS. [Source: “Auditory Beat Stimulation and its Effect on Cognition and Mood States; Chaieb, L, Wilpert, E. C., Reber, T., and Fell, J.; Front Psychiatry; 2015; 6:70].

Music therapy has also been studied respective of cognitive behavior for clients having severe mental illnesses. [Source: “Effect of the Group Music Therapy on Brain Wave, Behavior, and Cognitive Function among Clients with Chronic Schizophrenia; Kwon, R N, PhD, Kwon, M.; Gang, R N, PhD, M., Oh, R N, PhD, K., Asian Nursing Research, Vol. 7, Iss. 4, Dec. 2013, p. 168-174]. Notably, measurement of brain waves associated with alpha-rhythm sleep (e.g., any electroencephalography (“EEG”)-measured sleep with a frequency in the alpha range (8-13 Hz) [Source: https://www.sciencedirect.com/topics/nursing-and-health-professions/alpha-rhythm; Retrieved on: Jun. 1, 2019]) “reveal[ed] that the participants in the music therapy may have experienced more joyful emotions throughout the sessions.” [Source: “Effect of the Group Music Therapy on Brain Wave, Behavior, and Cognitive Function among Clients with Chronic Schizophrenia; Kwon, R N, PhD, Kwon, M.; Gang, R N, PhD, M., Oh, R N, PhD, K., Asian Nursing Research, Vol. 7, Iss. 4, December 2013, p. 168-174]. Nevertheless, further inquiry into the effect of music on targeting other wave-states of sleep, e.g., theta frequencies, remains largely unstudied.

In view of the foregoing, it is clear that these traditional techniques are not perfect and leave room for more optimal approaches.

### BRIEF SUMMARY OF THE INVENTION

In one aspect, the present invention is a platform and system to help the health care services providers to provide health care services 24 hours and providing client mental health assessment and treatment systems addressing the root causes and triggers of negative emotional states or conditions

In another aspect, a client accesses his account on the system to choose mental health care services comprising stress/anxiety tests, guided meditations, video therapies, exercise routines, nutrition Log, task Management, groups, challenges, and, chat.

In another aspect, a client will answer a series of questions to determine the client's mental state, and recommendations are generated for treatments that could help relieve some of these negative feelings, these recommendations include relevant contact information, advice, guided meditations and video therapies.

In another aspect, there are guided meditations wherein the system integrates subliminal isochronic tones to aid treatment and achieve results, where there are guided meditations covering a variety of topics and emotional states a client may be going through stress relief, depression, confidence, motivation or various fears.

In another aspect, there are video therapies wherein the system provide therapies and treatments accessible to clients at any time to enable clients to continue their healing any time of the day, access educational classes and motivational speeches.

In another aspect there are exercise routines provided to clients at any time to enable clients to continue their healing any time of the day.

In another aspect there is a nutrition Log wherein the system provide nutrition information accessible to clients at any time to enable clients to eat healthy.

In another aspect, the present invention includes task Management software which helps clients address overwhelming mental states and find stress relief outside the mental health care services provider clinic, this process begins by explaining how the mind processes and stores information, and how these thoughts when compiled can create stress, the services provider of healthcare will teach client how to organize these thoughts by having them write down the first five tasks that come to mind, prioritizing them, and allowing them to mark them off one by one as completed, clients are encouraged to remain concise and actionable by limiting the input character count of each task.

In another aspect, the present invention includes groups wherein clients can build communities of users with similar goals and interests, and challenges the user can personalize to inspire themselves and other users to stay on track with their treatment and recovery.

In another aspect, the present invention includes challenges option to let clients challenge each other to inspire themselves to stay on track with their treatment and recovery.

In another aspect, the present invention includes chat to help clients reach out to the services providers of mental health care at any time with questions or for advice.

In another aspect, the present invention helps services providers of mental health care provide in-house programs to provide their clients supplemental treatments that would help.

In another aspect the present invention helps the services providers of mental health care offer unlimited wellness, mental health care services and personal development services to their clients by providing treatments they can access anywhere.

In another aspect the present invention provides testing of negative emotional states resulting in individualized therapeutic solutions.

In another aspect the present invention helps services providers of mental health care to enable a client to take what they learn in a clinical environment and practice it on a daily basis, and when negative emotional states arise, those that may trigger unwanted outcomes such as relapse, they can address the root cause of it immediately through mobile therapy access: task management for overwhelming states, tests to determine a personalized therapy plan, meditations integrating isochronic waves to achieve ideal mindset for therapy to succeed, in this model, more important than constantly medicating is addressing the root emotional issues and triggers that lead to the unwanted outcomes the client is trying to overcome addiction.

### DESCRIPTION OF THE DRAWINGS

In order to best describe the manner in which the above-described embodiments are implemented, as well as define other advantages and features of the disclosure, a more particular description is provided below and is illustrated in the appended drawings. Understanding that these drawings depict only exemplary embodiments of the invention and are not therefore to be considered to be limiting in scope, the examples will be described and explained with additional specificity and detail through the use of the accompanying drawings in which:

FIG. 1 illustrates an example overview flow diagram for automatic client mental health state detection, assessment, classification, testing and remote treatment delivery in accordance with an embodiment of the present invention;

FIG. 2 illustrates an example flow diagram for month-by-month treatment of a client with treatment options for specific mental states in accordance with an embodiment of the present invention;

FIG. 3 illustrates an example flow diagram of a computer-based system implementing multiple modules directed to automatic client mental health state detection, assessment, classification testing and remote isochronic sound treatment delivery in accordance with an embodiment of the present invention;

FIG. 4 illustrates an example flow diagram for electronically providing a questionnaire for client completion regarding the automated prescription of a corresponding treatment method in accordance with an embodiment of the present invention;

FIG. 5 illustrates an example flow diagram of a computer-based method for automated client mood detection for treatment method identification targeting particular brain waves in accordance with an embodiment of the present invention;

FIG. 6 illustrates an example biofeedback loop for sensor and computer-based measurement of the brain's electrical and physiological activity in accordance with an embodiment of the present invention;

FIG. 7 illustrates an example guided user interface (“GUI”) environment implemented in a portable electronic peripheral device for automatic client mental health state detection, assessment, classification, testing and remote treatment delivery in accordance with an embodiment of the present invention;

FIG. 8 is a block diagram depicting an example client/server system that may be used by an example web-enabled/networked embodiment of the present invention; and

FIG. 9 illustrates a block diagram depicting an example client/server communication system that may be used for automatic remote client mental health detection, assessment, and treatment in accordance with an embodiment of the present invention.

FIG. 10 shows healthcare companies and how they can use it for their clients.

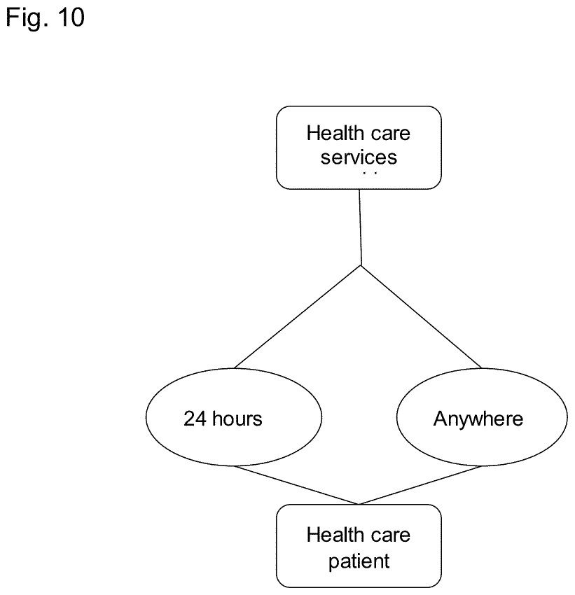

FIG. 11 shows individual business that can use it for their clients.

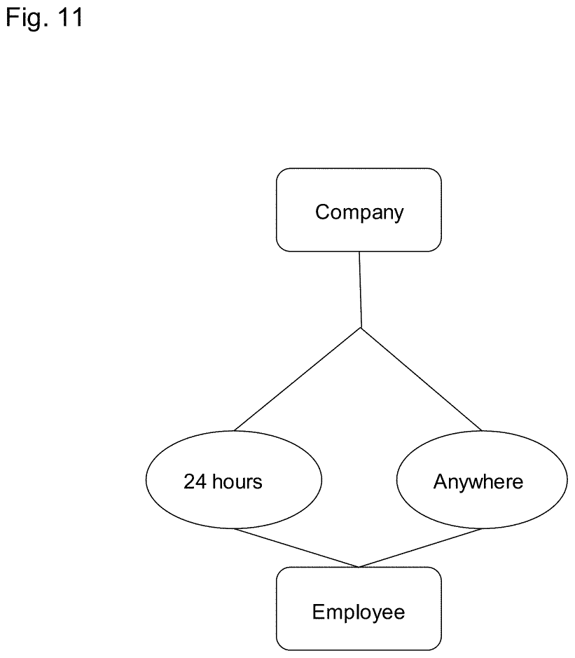

FIG. 12 shows that an individual can use the system for personal development as well.

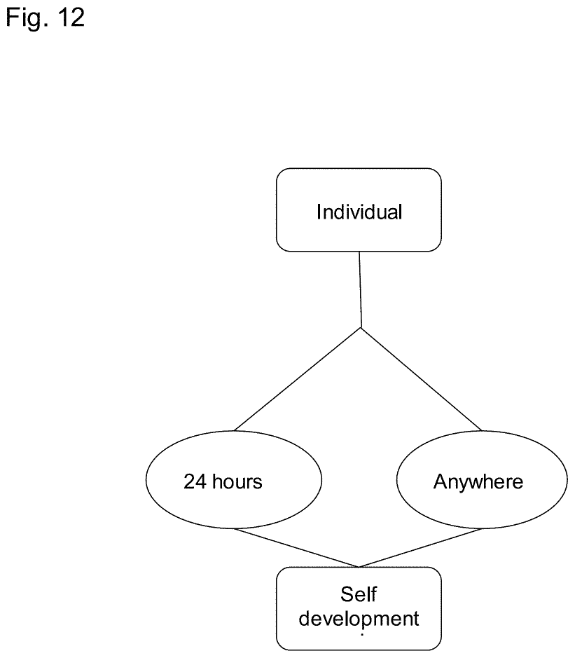

It should be noted that the accompanying figures are intended to present illustrations of few exemplary embodiments of the present disclosure. These figures are not intended to limit the scope of the present disclosure. It should also be noted that accompanying figures are not necessarily drawn to scale.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Reference will now be made in detail to selected embodiments of the present disclosure in conjunction with accompanying figures. The embodiments described herein are not intended to limit the scope of the disclosure, and the present disclosure should not be construed as limited to the embodiments described. This disclosure may be embodied in different forms without departing from the scope and spirit of the disclosure. It should be understood that the accompanying figures are intended and provided to illustrate embodiments of the disclosure described below and are not necessarily drawn to scale. In the drawings, like numbers refer to like elements throughout, and thicknesses and dimensions of some components may be exaggerated for providing better clarity and ease of understanding.

Moreover, although the following description contains many specifics for the purposes of illustration, anyone skilled in the art will appreciate that many variations and/or alterations to said details are within the scope of the present technology. Similarly, although many of the features of the present technology are described in terms of each other, or in conjunction with each other, one skilled in the art will appreciate that many of these features can be provided independently of other features. Accordingly, this description of the present technology is set forth without any loss of generality to, and without imposing limitations upon, the present technology.

It should be noted that the terms “first”, “second”, and the like, herein do not denote any order, ranking, quantity, or importance, but rather are used to distinguish one element from another. Further, the terms “a” and “an” herein do not denote a limitation of quantity, but rather denote the presence of at least one of the referenced item.

### General Techniques

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14. (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical low.” The term “substantial” is a meaningful modifier implying “approximate,” rather than “perfect” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s ma include without limitation, an individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

### Definitions

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It Is further contemplated that those skilled in the art will readily recognize the practical situations where such living makers, users, and/or participants with embodiments of the present invention may be in whole, or in part, replaced with such non-living makers, users, and/or participants with embodiments of the present invention.

Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

References to “trainer”, “mental health trainer”, or “behavioral health trainer” and/or “instructor” implies a skilled provider of behavioral services for clients in accordance with established treatment plans. “NLP” refers to a neuro-linguistic programming, inclusive of qualified NLP practitioners, related to identification of how the human mind handles, stores and retrieves information. NLP is an approach to communication, personal development, and psychotherapy, and claims that there is a connection between neurological processes (neuro-), language (linguistic) and behavioral patterns learned through experience (programming), and that these processes may be modified to work collectively to achieve specific goals in life. [Source: Tosey, P. & Mathison, J., (2006) “Introducing Neuro-Linguistic Programming.” Centre for Management Leaming & Development, School of Management, University of Surrey; Dilts, R., Grinder, J., Delozier, J., and Bandler, R. (1980). Neuro-Linguistic Programming: Volume I: The Study of the Structure of Subjective Experience. Cupertino, Calif.: Meta Publications. p. 2.]

Reference to “T.I.M.E.” techniques implies Time Integration for Maximum Empowerment. [Source: https://www.transformdestiny.com/what-is-time-techniques.asp; Retrieved on May 30, 2019]. T.I.M.E. techniques are “a collection of techniques for eliminating negative emotions, disempowering beliefs and limiting decisions tom the past, while creating and setting achievable, desirable goals to create a compelling future. TIME Techniques also addresses the way we perceive time, both semantically and neurologically.” [Source: https://www.transformdestiny.com/what-is-time-techniques.asp; Retrieved on May 30, 2019].

Reference to “meditation” implies a practice where an individual uses a technique—such as mindfulness, or focusing their mind on a particular object, thought or activity—to train attention and awareness, and achieve a mentally clear and emotionally calm and stable state. [Source: Walsh, Roger; Shapiro, Shauna L. (2006). “The meeting of meditative disciplines and western psychology: A mutually enriching dialogue”.

American Psychologist. 61 (3): 227-239; Rael Cahn; John Polich (2006). “Meditation states and traits: EEG, ERP, and neuroimaging studies”. Psychological Bulletin. 132 (2): 180-211; R. Jevning; R. K. Wallace; M. Beidebach (1992). “The physiology of meditation: A review: A wakeful hypometabolic integrated response”. Neuroscience & Biobehavioral Reviews. 16 (3): 415-24; “Definition of meditate”. Merriam-Webster Dictionary. 18 Dec. 2017. Retrieved 25 Dec. 2017; “meditate”. Oxford Dictionaries—English.]

Reference to “hypnotherapy” are generally intended to mean a type of complementary and alternative medicine in which the mind is used to help with a variety of problems, such as breaking bad habits or coping with stress. [Source: “Complementary and alternative medicine (CAM)”. National Health service. 2015-03-26. Retrieved 24 Jul. 2017.° ]

Reference to “emotional freedom techniques (“EFT”)” implies a form of counseling intervention that draws on various theories of alternative medicine including acupuncture, neuro-linguistic programming (“NLP”), energy medicine, and Thought Field Therapy (“TFT”). Advocates claim that the technique may be used to treat a wide variety of physical and psychological disorders, and as a simple form ofself-administered therapy. [Source: Craig, G (n.d.). EFT Manual (pdf). Retrieved 2011 May 3.]

Reference to “BioScan” implies a computer-based testing tool for diagnosing hidden illnesses. [Source: www.bioscan.com; Retrieved on: May 31, 2019].

Reference to “tai chi” implies an internal Chinese martial art practiced for both its defense training, its health benefits and meditation. The term taiji refers to a philosophy of the forces of yin and yang, related to the moves. Though originally conceived as a martial art, it is also typically practiced for a variety of other personal reasons: competitive wrestling in the format of pushing hands (tui shou), demonstration competitions and achieving greater longevity.

Reference to “yoga” implies a group of physical, mental, and spiritual practices or disciplines which originated in ancient India. Yoga is one of the six orthodox schools of Hindu philosophical traditions. [Source: Feuerstein, Georg (23 Oct. 2012). The Yoga Tradition: Its History, Literature, Philosophy and Practice. Hohm Press. There is a broad variety of yoga schools, practices, and goals in Hinduism, Buddhism, and Jainism. [Source: White, David Gordon (2011), Yoga, Brief History of an Idea (Chapter 1 of “Yoga in practice”) (PDF), Princeton University Press; Denise Lardner Carmody, John Carmody (1996), Serene Compassion. Oxford University Press US. p. 68; Stuart Ray Sarbacker, Samiidhi: The Numinous and Cessative in Inda-Tibetan Yoga. SUNY Press, 2005, pp. 1-2; Tattvarthasutra [6.1], see Manu Doshi (2007) Translation of Tattvarthasutra, Ahmedabad: Shrut Ratnakar p. 102]. The term “yoga” in the Western world often denotes a modern form of Hatha yoga, consisting largely of the postures called asanas (e.g., yogic postures and/or movements).

Reference to “chakra” implies the various focal points in the subtle body used in a variety of ancient meditation practices, collectively denominated as Tantra, or the esoteric or inner traditions of Hinduism. [Source: Chakra: Religion, Encyclopedia Britannica; Grimes, John A (1996). A Concise Dictionary of Indian Philosophy: Sanskrit Terms Defined in English. State University of New York Press. pp. 100-101; Lochtefeld, James G (2002). The Illustrated Encyclopedia of Hinduism: A-M. The Rosen Publishing Group. p. 137.]

Reference to “Time Line Therapy®” is generally intended to mean the latest development in neuro-linguistic Programming (“NLP”), Time Line Therapy® techniques are a unique method for creating change in business, education, and therapy. Time Line Therapy® uses a person's own internal “Time Line” to work with his or her unconscious mind in a variety of ways; including, healing emotional traumas and eradicating unwanted thoughts, emotions and behaviors. [Source: https://www.nlpcoaching.com/time-line-therapy/; Retrieved on: May 31, 2019].

Reference to “transform life” implies a self-transformation towards the maximization of one's true potential. [Source: https://www.jackcanfield.com/blog/5-simple-ways-to-transform-your-life/; Retrieved: May 31, 2019]].

Reference to “create your life now . . . , change your life now . . . , make your life now . . . ” implies a conscientious effort to make life changes for the good, forever. [Source: https://www.lifehack.org/310325/10-things-change-your-life-forever; Retrieved: May 31, 2019].

Reference to “stress management” implies a set of techniques and programs intended to help people deal more effectively with stress in their lives by analyzing the specific stressors and taking positive actions to minimize their effects. [Source: https://positivepsychologyprogram.com/stress-management-techniques-tips-burn-out/; Retrieved on: May 31, 2019].

Reference to “anxiety management” implies a program to treat anxiety (e.g., excessive nervousness, fear, apprehension, and worry) that may include exercise and a regular sleep schedule. [Source: https://www.webmd.com/anxiety-panic/anxiety-tips#1; Retrieved on: May 31, 2019].

Reference to “mindset” implies an established set of attitudes held by someone; a set of assumptions, methods, or notations held by one or more people or groups of people. A mindset can also be seen as incident of a person's world view or philosophy of life.

Reference to “motivational therapy (“MT”)” implies a combination of humanistic treatment and enhanced cognitive-behavioral strategies, designed to treat substance abuse. It is similar to motivational interviewing and motivational enhancement therapy.

Reference to “relaxation” implies the state of being free from tension and anxiety and/or the action of making a rule or restriction less strict.

Reference to “autogenic training” implies a desensitization-relaxation technique developed by the German psychiatrist Johannes Heinrich Schultz [source: LACI., SPENCER (2015). FLOTATION: a guide for sensory deprivation, relaxation, & isolation tanks] by which a psychophysiologically determined relaxation response is obtained. [Source: Stetter, Friedhelm; Kupper, Sirko (2002-03-01). “Autogenic Training: A Meta-Analysis of Clinical Outcome Studies”. Applied Psychophysiology and Biofeedback. 27 (1): 45-98.]

Reference to “overcome overwhelming states” implies a concerted effort to address symptoms of being overwhelmed, e.g., by negative emotions, such as anger, fear, anxiety, or guilt. [Source: https://www.talkspace.com/blog/feeling-overwhelmed/; Retrieved on: May 31, 2019].

Reference to “thoughts” implies an idea or opinion produced by thinking, or occurring suddenly in the mind.

Reference to “balance your energy” implies an effort to bring energy balance healing into your everyday life. [Source: https://www.mindbodygreen.com/0-22920/4-techniques-to-ground-heal-balance-your-energy-levels.html; Retrieved on: May 31, 2019].

Reference to “open your energy” implies a concerted effort to the opening of one's spiritual channels to have access to the metaphysical universe. [Source: https://www.drsha.com/open-your-spiritual-channels/; Retrieved on: May 31, 2019].

Reference to “open your blocks” implies a process to encourage the creative handling of mental stoppage points by re-engaging one's creativity. Steps may include: (1) stepping away from the situation; (2) writing or talking about the problem; and, (3) free write solutions.

Reference to “connect to spirit” implies an effort to communicate with metaphysical or supernatural entities that symbolically help seeking persons find wholeness. [Source: https://lonerwolf.com/connect-with-spirit-guides/; Retrieved on: 05-31-19].

Reference to “spirituality” implies the quality of being concerned with the human spirit or soul as opposed to material or physical things.

Reference to “spiritual healing” implies the activity of making a person healthy without using medicines or other physical methods, sometimes as part of a religious ceremony.

Reference to “healing” implies the process of making or becoming sound or healthy again.

Reference to “therapeutic touch (“TT”)” implies a pseudoscientific energy therapy which practitioners claim promotes healing and reduces pain and anxiety. [Source: Astin, J A; Harkness, E; Ernst, E (2000). “The efficacy of “distant healing”: A systematic review of randomized trials”. Annals of internal Medicine. 132 (11): 903-10.]

Reference to “how the mind works” implies the 1997 book (entitled the same) by Canadian-American cognitive scientist Steven Pinker, in which the author attempts to explain some of the human mind's poorly understood functions and quirks in evolutionary terms. [Source: Pinker, S. How the Mind Works (Norton, 1997) p. 50].

Reference to “unconscious mind” implies a reservoir of feelings, thoughts, urges, and memories that outside of our conscious awareness. Most of the contents of the unconscious are unacceptable or unpleasant, such as feelings of pain, anxiety, or conflict. [Source: https://www.verywellmind.com/what-is-the-unconscious-2796004; Retrieved on: May 31, 2019].

Reference to “conscious mind” includes such things as the sensations, perceptions, memories, feeling, and fantasies inside of our current awareness. Closely allied with the conscious mind is the preconscious, which includes the things that we are not thinking of at the moment but which we can easily draw into conscious awareness. [Source: https://www.verywellmind.com/what-is-the-conscious-mind-2794984; Retrieved on: May 31, 2019].

Reference to “post-traumatic stress disorder (“PTSD”)” implies a mental health condition that's triggered by a terrifying event—either experiencing it or witnessing it. Symptoms may include flashbacks, nightmares and severe anxiety, as well as uncontrollable thoughts about the event. [Source: https://www.mayoclinic.org/diseases-conditions/post-traumatic-stress-disorder/symptoms-causes/syc-20355967; Retrieved on: May 31, 2019].

Reference to “attention-deficit hyperactivity disorder (“ADHD”)” implies a condition with symptoms such as inattentiveness, impulsivity, and hyperactivity. The symptoms differ from person to person. ADHD was formerly called ADD, or attention deficit disorder. Both children and adults can have ADHD, but the symptoms always begin in childhood. Adults with ADHD may have trouble managing time, being organized, setting goals, and holding down a job. [Source: https://www.webmd.com/add-adhd/default.htm; Retrieved on: May 31, 2019].

Reference to “anger” implies an intense emotional state. It involves a strong uncomfortable and hostile response to a perceived provocation, hurt or threat. [Source: Videbeck, Sheila L. (2006). Psychiatric Mental Health Nursing (3rd ed.). Lippincott Williams & Wilkins; Retrieved on: May 31, 2019]. A person experiencing anger will often experience physical conditions, such as increased heart rate, elevated blood pressure, and increased levels of adrenaline and noradrenaline. [Source: “Anger definition”. Medicine.net. Retrieved 2008 Apr. 5.] Anger has been viewed as an emotion which triggers part of the fight or flight brain response. Harris, W., Schoenfeld, C. D., Gwynne, P. W., Weissler, A. M., Circulatory and humoral responses to fear and anger, The Physiologist, 1964, 7, 155.]

Reference to “anxiety and panic attacks” implies intense periods of fear or feelings of doom developing over a very short time frame—up to 10 minutes—and associated with at least four of the following: sudden overwhelming fear; palpitations; sweating; trembling shortness of breath; sense of choking; chest pain; nausea, dizziness; a feeling of being detached from the world (de-realization); fear of dying; numbness or tingling in the limbs or entire body; and/or chills or hot flashes. [Source: https://www.webmd.com/anxiety-panic/guide/anxiety-attack-symptoms; Retrieved on: May 31, 2019].

Reference to “bipolar disorder” implies a brain disorder that causes unusual shifts in mood, energy, activity levels, and the ability to carry out day-to-day tasks. There are four basic types of bipolar disorder; all of them involve clear changes in mood, energy, and activity levels. These moods range from periods of extremely “up,” elated, and energized behavior (known as manic episodes) to very sad, “down,” or hopeless periods (known as depressive episodes). Less severe manic periods are known as hypomanic episodes. [Source: https://www.nimh.nih.gov/health/topics/bipolar-disorder/index.shtml; Retrieved on: May 31, 2019].

Reference to “body dysmorphic disorder” implies a mental disorder in which one can't stop thinking about one or more perceived defects or flaws in his or her appearance—a flaw that, to others, is either minor or not observable. But the affected individual may feel so ashamed and anxious that he or she may avoid many social situations. [Source: https://www.mayoclinic.org/diseases-conditions/body-dysmorphic-disorder/symptoms-causes/syc-20353938; Retrieved on: May 31, 2019].

Reference to “depression” implies a state of low mood and aversion to activity. It can affect a person's thoughts, behavior, motivation, feelings, and sense of well-being. It may feature sadness, difficulty in thinking and concentration and a significant increase/decrease in appetite and time spent sleeping, and people experiencing depression may have feelings of dejection, hopelessness and, sometimes, suicidal thoughts. It can either be short term or long term. [Source: de Zwart, P. L.; et al. (2018). “Empirical evidence for definitions of episode, remission, recovery, relapse and recurrence in depression: a systematic review”. Epidemiology and Psychiatric Sciences: 1-19.]

Reference to “dissociative disorders” implies mental disorders that involve experiencing a disconnection and lack of continuity between thoughts, memories, surroundings, actions and identity. People with dissociative disorders escape reality in ways that are involuntary and unhealthy and cause problems with functioning in everyday life. [Source: https://www.mayoclinic.org/diseases-conditions/dissociative-disorders/symptoms-causes/syc-20355215; Retrieved on: May 31, 2019].

Reference to “recreational drugs and alcohol” implies the use of a psychoactive drug to induce an altered state of consciousness for pleasure, by modifying the perceptions, feelings, and emotions of the user. When a psychoactive drug enters the user's body, it induces an intoxicating effect. Generally, recreational drugs are in three categories: depressants (drugs that induce a feeling of relaxation and calm); stimulants (drugs that induce a sense of energy and alertness); and hallucinogens (drugs that induce perceptual distortions such as hallucination).

Reference to “obsessive compulsive disorder (“OCD”)” implies a mental disorder in which a person feels the need to perform certain routines repeatedly (called “compulsions”), or has certain thoughts repeatedly (called “obsessions”). [Source: The National Institute of Mental Health (NIMH) (January 2016). “What is Obsessive-Compulsive Disorder (OCD)?”. U.S. National Institutes of Health (NIH). Archived from the original on 23 Jul. 2016; Retrieved 24 Jul. 2016.]

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open—ended and mean “including but not limited to”. When employed in the appended claims, this term does not foreclose additional-structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the Mechanisms/unit/circuit/component can be said to be configured to (or be operable) for perform(ing) the task even when the specified mechanisms/unit/circuit/component is not currently operational (e.g., is not on). The mechanisms/units/circuits/components used With the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

While embodiments herein may be discussed in terms of a processor having a certain number of bit instructions/data, those skilled in the art will know others that may be suitable such as 16 bits, 32 bits, 64 bits, 128 s or 256-bit processors or processing, which can usually alternatively be used. Where a specified logical sense is used, the opposite logical sense is also intended to be encompassed.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any-type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as web pages.

Websites comprise a collection of connected, or otherwise related, web pages. The combination of all the websites and their corresponding web pages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention.

It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order: The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor.

Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable., twisted pair, optical fiber, waveguides, etc.) and/or wireless connections (e.g., radio frequency waveforms, free-space optical waveforms, acoustic waveforms, etc.). Examples of a network may include: an Internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, Firefox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either.

The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium.

Combinations of the above should also be included within the scope of the computer-readable media. While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

### Introduction

Social stigma and public perceptions against mental health and related issues often prevents individuals in need of medical or psychological attention from receiving attention and care in times of need. Many people who would otherwise benefit from access and receipt of mental health services choose not to pursue such help or fail to adequately participate once treatment has begun. [Source: “How Stigma Interferes with Mental Health Care”; Corrigan, P.; Univ. of Chicago; American Psychologist; Vol. 59, No. 7, 614-625]. Stigma against those facing mental health issues is a major reason why sick persons fail to actively seek the help they need, largely to avoid the label of mental illness and the harm it often brings. Such stigma can bring about two types of harm: (1) diminishment of self-esteem of the disaffected individual; and, (2) deprivation of social interaction and opportunities with others.

Trends regarding mentally sick persons proactively seeking appropriate help are disconcerting. Notably, many people with mental illness never pursue treatment in the first place, while others who begin soon fail to fully adhere to services as prescribed. Research from the Epidemiologic Catchment Area (“ECA”) Study showed that less than 30% of people with psychiatric disorders seek treatment. [Source: Regier et al., 1993]. A subsequent large-scale study, the National Comorbity Survey, showed similar results. [Source: Kesseler et al., 2001]. Less than 40% of respondents with mental illness in the past year received stable treatment.

Before being able to comprehensively address mental health issues, society must first fully understand why people with mental health problems fail to engage in treatment. Stigma appears as the relevant factor and may be further sub-categorized into four social-cognitive processes: cues, stereotypes, prejudice, and discrimination. [Source: “How Stigma Interferes with Mental Health Care”; Corrigan, P.; Univ. of Chicago; American Psychologist; Vol. 59, No. 7, 615]. Negative stereotypes, prejudice and discrimination about people with mental illnesses include violence (e.g., people with mental illness are dangerous), incompetence (e.g., people with mental illness are incapable of independent living or real work), and blame (e.g., because of character deficiencies, people with mental illness are themselves responsible for the onset and continuation of their disorders). [Source: “How Stigma Interferes with Mental Health Care”; Corrigan, P.; Univ. of Chicago; American Psychologist; Vol. 59, No. 7, 616].

Thus, there is a clear need for therapeutic methods allowing for mentally ill persons to readily access medical care and psychological counseling, if needed, discretely, to avoid facing such stigma as described above. This needed discretion may be made possible via rapid advances in modem computing technology, which permit for around-the-clock communication access, allowing psychiatrists, psychologists, social workers, therapists, and other healthcare and mental wellbeing professionals to remain in regular communication with clients.

However, although current methods and techniques can accommodate various parts of mental health wellbeing and mental illness treatment, no single comprehensive computer-based application (“app”) exists for automatic client mental health state detection, assessment, classification, testing and remote treatment delivery Thus, the disclosed embodiments seek to, at a minimum, address this unmet need by offering an integrated treatment plan featuring the automatic detection and classification of the mental state or condition of a remotely-located client. The app also provides testing to administer therapy commensurate with the outcome of the testing, such therapy being inclusive of isochronic sounds or tones, which have shown improvements in focus, relaxation, energy levels, sleep and more without the requirement of additional drugs or special equipment. [Source: https://www.mindamend.com/brainwave-entrainment/isochronic-tones/; Retrieved: 06-01-19].

Such isochronic sounds or tones are generally understood to be a fast and effective audio-based way to stimulate various areas of the brain by guiding dominant brainwave activity to different frequencies resulting in a change in the mental state of the listener. Isochronic sounds or tones can also be used in different ways to achieve different benefits, most popular uses extending to improvements in studying, focus and concentration in particular by guiding the brain to a preferred or optimum mental state. Isochronic sounds or tones can be used for at least the following example purposes: stress or anxiety relief, meditation and relaxation, increases in focus, cognition and memory, improving sleep and for power napping, and energy and motivation.

The app and related process for using it allows various mental health professionals such as hypnotherapists, NLP, EFT, TIME techniques practitioners, and masseuses to establish relationships to deliver isochronic sounds or tones to accordingly treat mental clients. Such relationships can be established on a contractual basis with hospital, clinics, doctors, medical offices/clinics, rural and urban treatment centers and other similar groups or entities (referred to herein as “medical facilities”) to allow the mental health professionals to provide supplemental client treatment services to medical facilities.

As introduced above, “it has been estimated that 75-90 percent of all visits to primary care physicians are for stress related problems.” [Source: https://www.stress.org/arnericas-1-health-problem; Retrieved on May 30, 2019; Job Stress: America's Leading Adult Health Problem, by Paul J. Rosch, M.D., F.A.C.P., in USA Magazine, May 1991; American Academy of Family Physicians Survey, 1988, U.S. News & World Report, Dec. 11, 1995; also, Research by Perkins (1994) cited in the Harvard Business Review showed that 60% to 90% of doctor visits were stress-related.] Such visits may include, but are not limited to, numerous feelings and belief systems related causes such as: depression, fears, anger, sadness, addiction, and phobias.

Medical facilities may not all be adequately equipped to provide mental health clients needed supplemental treatment to alleviate stress and all related emotional triggers thereof. Such triggers can also cause other negative and undesirable behaviors such as drug and controlled substance addiction. Using the app to establish an in-house education and treatment outlet for professionals assists clients better cope with negative moods and emotions (e.g., stress and anxiety) by providing an automatic, electronically-based, readily available and accessible solution. Clients will also, as a result of the supplemental treatment they receive through the app, be better positioned to benefit from the primary medical treatment they are concurrently receiving.

### General Description of the Disclosed Embodiments

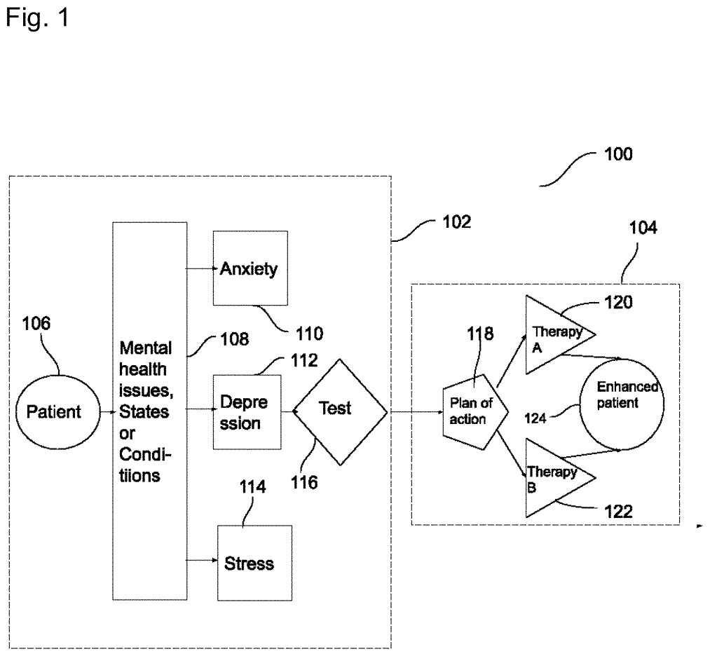

FIG. 1 illustrates an example overview flow diagram for automatic client mental health state detection, assessment, classification, testing and remote treatment delivery in accordance with an embodiment of the present invention. Client treatment system 100, executed at least in part on a computer, e.g., requiring storage in the non-transitory memory of the computer, comprises at least a detection module 102 and a treatment module 104. In an embodiment, the client treatment system 100 can be implemented as a website to be accessed via the Internet and/or as a mobile application, referred to as an “app.” Upon access of the client treatment system 100, a client 106 can interact with or otherwise be considered to generally be a part of the detection module 102. The client 106 can seek supplemental treatment assistance from the client treatment system 100 to support a primary or principal treatment plan provided by, for example, a private or government medical facility for any and all treatments related to addressing the root cause, or causes, of, for example, the mental health issue, problem, concern, or illness of the client. Such root causes can be related to, for example (and without limitation), physical, spiritual, emotional, and/or belief-oriented factors, e.g., stress, anxiety, depression, fear, phobia, drug and/or controlled substance addiction, compulsive behaviors, etc.

Medical facilities can enhance treatments offered to clients by integrating usage of client treatment system 100, since many such medical facilities may not be adequately equipped to provide supplemental mental healthcare related services, such as follow-up psychological counseling, dietary monitoring, establishment and maintenance of a strict exercise regimen, etc. Such supplemental or ancillary treatments may also include (but are not limited to), stress relief classes, workshops, meditations, autogenic stress relief, diet, group mental health issue corrective therapy sessions, individual sessions, EFT, and NLP. These services can be provided by professionals, licensed, certified, or non-licensed, and may include any one or more selected from a group comprising: psychiatrists, psychologists, psychotherapists, hypnotherapists, cognitive behavioral therapists (“CBT”), emotional freedom technique (“EFT”) therapists, personal trainers (“PT”), NLP, medical doctors (“MD”), registered nurses (“RN”). Such medical and mental health professionals can provide services on as as-needed basis, both automatically and remotely, to one or more mental health clients contractually through the client treatment system 100, where such a contractual relationship can be less resource-intensive, e.g., expensive, than hiring and managing any one or more of these discussed supplemental mental health treatment programs with a client. The client treatment system 100 is envisioned to extend capabilities including, but not limited to, providing workshops, classes, one-on-one sessions, medication direction, exercise programs, and nutrition programs.

Returning to FIG. 1, the client 106 can interact with the client treatment system 100 through a variety of means, either directly or indirectly. In an example embodiment, the client 106 can physically touch a touch-screen active computer-based electronic peripheral device, e.g., a “smartphone” such as an apple iPhone®, to insert key information therein, e.g., to be stored locally on the non-transitory memory of the device and be electronically associated with client treatment system 100. The client 106, in an example embodiment, can also verbally speak out commands or phrases to be detected via voice-recognition features and capabilities of the smartphone for interaction and data entry or input into the client treatment system 100. The client 106, in an example embodiment, can also wear and/or otherwise be prepared with electrodes, sensors, and/or other biometric feedback equipment to electronically, via wired or wireless devices and systems, communicate health and/or brain related data to the client treatment system 100. One skilled in the art will appreciate that this example listing of electronic, computer-based, telecommunications methodology is non-exhaustive and that other suitable means for obtaining and/or extracting and communicating data or information related to client mental health to the client treatment system 100 may be implemented without departing from the scope and spirit of the disclosed embodiments.

Through the various input methodologies discussed above, or other suitable methodologies, the client 106 inputs his or her data or information concerning mental health issues, states or conditions at client mental health input step 108. Such input may be visual as provided by the client treatment system 100 installed in a smartphone, e.g., offering one or more touch-sensitive representations of icons which, upon contract therewith, trigger a corresponding communication to the client treatment system 100 regarding the input mental state or condition of the client 106. In an embodiment, such communications may be accomplished via voice recognition capabilities of the computer-based portable electronic peripheral having the client treatment system 100 installed therein. The client mental health input step 108, upon at least partial completion, can parse input data or information into one or more distinct and separate categories corresponding to the particular mental health related mood, state, or condition of the client 106, e.g., (but not limited to) anxiety module 110, depression module 112, and/or stress module 114.

As conventionally understood, the anxiety 110 module refers to an emotion characterized by an unpleasant state of inner turmoil, often accompanied by nervous behavior such as pacing back and forth, somatic complaints (“butterflies in the stomach”), and rumination. [Source: Seligman M E, Walker E F, Rosenhan D L. Abnormal psychology (4th ed.). New York: W. W. Norton & Company.] Anxiety, as so defined, indudes subjectively unpleasant feelings of dread over anticipated events, such as the feeling of imminent death. [Source: Davison G C (2008). Abnormal Psychology. Toronto: Veronica Visentin. p. 154.] It is often accompanied by muscular tension [Source: American Psychiatric Association (2013). Diagnostic and Statistical Manual of Mental Disorders (Fifth ed.). Arlington, Va.: American Psychiatric Publishing. p. 189], restlessness, fatigue and problems in concentration.

As conventionally understood, the depression module 112 refers to a state of low mood and aversion to activity. Depression can affect a person's thoughts, behavior, motivation, feelings, and sense of well-being. It may feature sadness, difficulty in thinking and concentration and a significant increase/decrease in appetite and time spent sleeping, and people experiencing depression may have feelings of dejection, hopelessness and, sometimes, suicidal thoughts. It can either be short term or long term. [Source: de Zwart, P. L.; et al. (2018). “Empirical evidence for definitions of episode, remission, recovery, relapse and recurrence in depression: a systematic review”. Epidemiology and Psychiatric Sciences: 1-19.]

As conventionally understood, the stress module 114 can refer to psychological stress defined as a feeling of strain and pressure. [Source: “Stress”. Mental Health America. 2013 Nov. 18. Retrieved 2018 Oct. 1.] Small amounts of stress may be desired, beneficial, and even healthy. Positive stress helps improve athletic performance. It also plays a factor in motivation, adaptation, and reaction to the environment. Excessive amounts of stress, however, may lead to bodily harm. Stress can increase the risk of strokes, heart attacks, ulcers, and mental illnesses such as depression. [Source: Sapolsky, Robert M. (2004). Why Zebras Don't Get Ulcers. 175 Fifth Ave, New York, N.Y.: St. Martins Press. pp. 37, 71, 92, 271.] The stress module 114 can also refer to, in addition to or in the alternative to the definition provided above for psychological stress, physiological or biological stress, defined as an organism's response to a stressor such as an environmental condition. Stress is the body's method of reacting to a condition such as a threat, challenge or physical and psychological barrier. Stimuli that alter an organism's environment are responded to by multiple systems in the body. The autonomic nervous system and hypothalamic-pituitary-adrenal (HPA) axis are two major systems that respond to stress. [Source: Ulrich-Lai, Yvonne M.; Herman, James P. (7 Feb. 2017). “Neural Regulation of Endocrine and Autonomic Stress Responses”. Nature Reviews Neuroscience. 10 (6): 397-409.] One skilled in the art will appreciate that other suitable definitions or understandings of stress may exist without departing from the scope and spirit of the disclosed embodiments.

Returning to the functionality of the client mental health input step 108, the data input by the client 106 is classified into one or more categories such as the anxiety module 110, etc. One skilled in the art will appreciate that additional such modules may exist to accommodate various client moods and mental illnesses without departing from the scope and spirit of the disclosed embodiments. Upon the client treatment system 100 classifying the mental health issues, states, or conditions at the client mental health input step 108 into any one or more of, for example, the anxiety module 110, etc., the client treatment system 100 requests or requires the client 106 to take one or more tests at a test step 116 to determine personalized therapy options to address the current emotional state of the client 106.

The test step 116 can include, in an example embodiment, a series of targeted questions to determine how the client 106 is feeling, e.g., to determine whether the client 106 is depressed, overwhelmed, sad, stressed, anxious, and so on and so forth. Each question posed in the test step 116, in an embodiment, can include four responses, each response of the four responses being scaled or weighted. The client treatment system 100 totals the weighted response of each of the four responses to calculate the severity of the mental state or condition of the client 106 to generate corresponding recommendations commensurate for each mood or mental state of the client 106. That is, should the client 106 input data or information into the client treatment system 100 indicative of anxiety in the client mental health input step 108 for classification and direction to the anxiety module 110, the test 116 will generate and provide anxiety-related questioning to the client 106 to further determine the extent or severity of the anxiety to generate at least one plan of action step 118. This plan of action step 118 will generate options, suggestions, choices and/or required courses of action commensurate with the calculated degree of severity of the mental health of the client 106 calculated at the test step 116. Recommendations that can be provided at the plan of action step 118 include (but are not limited to): providing the contact information of relevant mental health professionals, advice, guidance, guided meditations, video therapies, and more.

Such recommendations provided by the plan of action step 118 represent a transition from the detection module 102 to the treatment module 104 of the client treatment system 100, and can be further classified into therapies including representative therapy A 120 and representative therapy B 120. One skilled in the art will appreciate that additional representative therapies C, D, . . . etc. may be suggested by the plan of action step 118 in response to the calculations or outcomes of the test step 116 without departing from the scope and spirit of the disclosed embodiments. In an example embodiment, the plan of action step 118 can suggest guided meditations, video therapies, and task management as any one or more of the therapy step A 120 and/or the therapy step B 122.

Guided meditations, as generally understood, refer to the integration of subliminal isochronic sounds, tones and/or waves to aid in mental health treatment of the client 106 to achieve favorable results, e.g., defined as the alleviation or the at least partial improvement of the mood, state or condition of the client 106. Guide meditations can cover a variety of topics and emotional states that the client 106 may be experiencing inclusive of (but not limited to): stress relief, depression, confidence, motivation, various fears, and so on and so forth.

Video therapies, as generally understood, are accessible to the client 106 via the client treatment system 100 at any time of the day or night, all days of the week, featuring pre-recorded video messages without live interaction or assistance. Such availability permits the client 106 to continue healing at any time of the day or night. Educational classes and motivational speeches are also offered as a part of the video therapies, as are additional certifications and therapies encouraging the client 106 to enhance his or her own capabilities regarding self-healing and recovery as well.

Task management, as generally understood, refers to a therapeutic method offered by the client treatment system 100 geared to assist the client 106 address and overcome overwhelming mental moods, states and/or conditions to find relief outside of a traditional primary care medical setting. The task management process begins with an explanation to the client 106 from the client treatment system 100 about how the human brain and/or mind processes and stores information, and how these thoughts, when compiled in certain ways, can create negative mood states such as (but not limited to) stress and anxiety. Task management teaches the client 106 how to organize his or her thoughts by, in an example embodiment, having the client 106 write down (e.g., on a piece of paper separate from the smartphone running the client treatment system 100 app) the first five tasks that come to his or her mind, prioritizing the tasks, and allowing the client 106 to mark the tasks off one by one upon completion. The client is encouraged, by the task management functionality as suggested or offered by the plan of action step 118 and/or the suggested therapies A, B 120, 122, respectively, to remain concise, precise and actionable by limiting the input character count to a pre-defined quantity, e.g., 150 to 400 characters, for each task.

Within the task management functionality as described here, in an embodiment, a real-time live chat section can also be offered. The chat section permits the client 106 to, within certain pre-specified hours, such as during normal business hours or upon a pre-scheduled consultation time, communicate with relevant professionals at, for example, a mental health care facility or institution, inclusive of traditional hospitals, clinic, psychiatric care wards, counseling centers, substance abuse and recovery centers, and so on and so forth. Also, in an example embodiment, the chat section may be confi_{gu}red to accommodate communication between multiple clients 106 amongst themselves to openly discuss common mental health topics and issues and methods of recovery. Moreover, in an example embodiment, the chat section can also provide for the creation and/or maintenance of social groups that the client 106 can build or otherwise interact with to access and communicate with communities of users with similar goals and interests. Such communication within the communities may serve to inspire the client 106 to stay on track for a complete recovery from any supplemental mental health treatment prescribed or suggested by the client treatment system 100. Clinics, hospitals, and other mental health care and service providers, in an embodiment, have the option, through the social groups, to particularize the therapies and tools offered to the client 106 from, for example, an access portal or dashboard to contribute their own videos, audio files, therapies and software specific to their respective clinic, etc.

Completion of the various steps listen from client mental health input step 108 to administration of the therapies A, B, 120, 122, respectively, results in an enhanced client 124, who is substantially healed, cured, corrected, or otherwise relieved of mental illness, ailment, concern, or issue. Overall, the client treatment system 100 seeks to focus on holistic mental health treatment therapy addressing the root cause or causes and triggers of negative emotional status of the client 106, rather than solely requiring the client 106 to receive primary care from, for example, a psychiatrist involving drug-based medication. The client treatment system 100 differs significantly from traditional treatment and therapeutic methods in providing convenient around-the-clock continued mobile-based access, integration of music-based therapies inclusive of the provision of isochronic sound waves, sounds and/or tones to aid in therapy, and particularized therapy offered commensurate to the type and severity of the mental ailment or illness of the client 106. Aspects of the disclosed embodiments of the client treatment system 100 include any-time therapy access, recovery process acceleration through extended treatment opportunities, therapy access outside of traditional hospital or clinic settings, e.g., where many of the triggering issues may occur, convenient mobile-based app access, and addressing of mental health problems and issues as they occur, rather than reactively after an episode, targeting the root cause or causes in real-time.

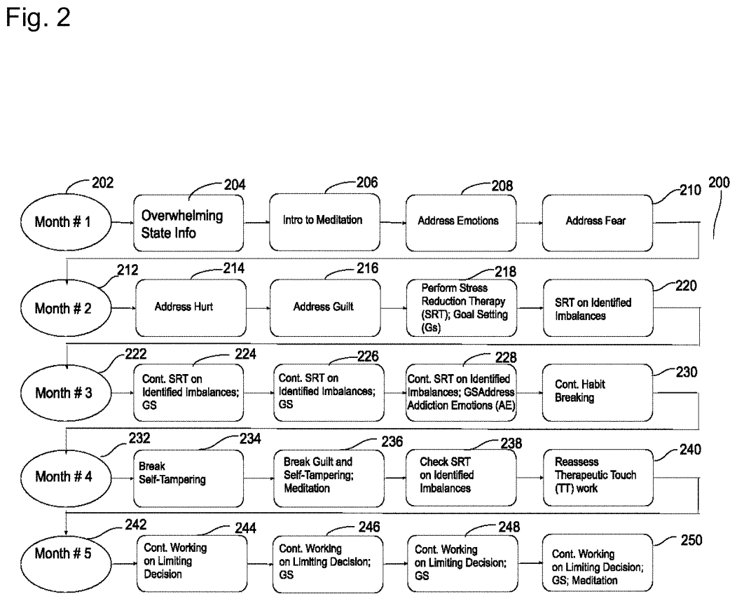

FIG. 2 illustrates an example counselor process flow chart with a counseling system 200 that, in an embodiment, can be practiced as a part of the therapies A, B, 120, 122 as recommended by the plan of action step 118 in the treatment module 104 of the client treatment system 100 shown in FIG. 1. The therapies A, B, 120, 122 can include monthly step-by-step treatment methodologies, outlined by steps 202-250 of the counseling system 200. Initially, in a first month 202, the client 106 can be assigned to provide mental state information, e.g., feeling overwhelmed, and to be assigned a task for writing 5 thoughts in a step 204. Also, in the step 204, the client can be introduced to therapeutic touch (“TT”) therapy, commonly understood and defined herein as a holistic, evidence-based therapy that incorporates the intentional and compassionate use of universal energy to promote balance and well-being. TT is generally understood to be a consciously directed process of energy exchange during which the practitioner uses the hands as a focus to facilitate the process. [Source: http://therapeutictouch.org/what-is-tt/; Retrieved on: Jun. 2, 2019]. The step 204 leads to a step 206 involving another phase of TTT therapy and an introduction to meditation, referring to a practice where an individual uses a technique—such as mindfulness, or focusing their mind on a particular object, thought or activity—to train attention and awareness, and achieve a mentally clear and emotionally calm and stable state. [Source: Walsh, Roger; Shapiro, Shauna L. (2006). “The meeting of meditative disciplines and western psychology: A mutually enriching dialogue”. American Psychologist. 61 (3): 227-239.] Next, TT therapy is continued in a step 210 from the step 208, where fears are also addressed. In a second month 212, TT therapy is continued in a step 214, which addresses hurt, prior to a step 216 for addressing guilt. A subsequent step 218 performs stress reduction therapy (SRT) and involves testing to identify mental imbalance. Goal setting is also initiated at the step 218, prior to performing SRT on the identified mental imbalances at step 220. In a third month 222, SRT is continued on the identified imbalances and goal setting is continued in a step 224 which leads to the additional sessions of the same in a step 226. TT therapy is continued and combined with work on addiction emotions (“AE”) such as anger and sadness, and work is initiated on habit breaking at a next step 228. Later, TT therapy is continued as is ongoing work on addressing AE and work on habit breaking in a step 230. In a fourth month 232, touch therapy is continued as is work on habit breaking and self-tampering in a step 234. Next, in a step 236, guilt is broken via continued guilt and self-tampering breaking. The results of SRT on the identified imbalances are checked in a following step 238 to recommend to a professional, e.g., psychiatrist, to taper off treatment as needed. TT therapy work is reassessed, as is limiting decision therapy, GS and meditation as needed in a following step 240. In a fifth month 242, TT is continued on limiting decision as needed in a step 244, which leads to a step 246 where TT is continued, as is work on limiting decisions and GS. TT therapy is further continued in a step 248, where decisions are further limited. Lastly, TT therapy is continued as is work on limiting decisions and GS at a step 250. One skilled in the art will appreciate that the number, sequence, and organization of the various steps 204-250 are provided for example purposes only and that other suitable configurations may exist without departing from the scope and spirit of the disclosed embodiments. Further, fewer or additional steps may be employed in the counseling system 200, and application of the counseling system 200 may be discontinued after completion of the fifth month 242 if deemed appropriate by, for example, a mental health professional and/or the client 106.

### System Structure

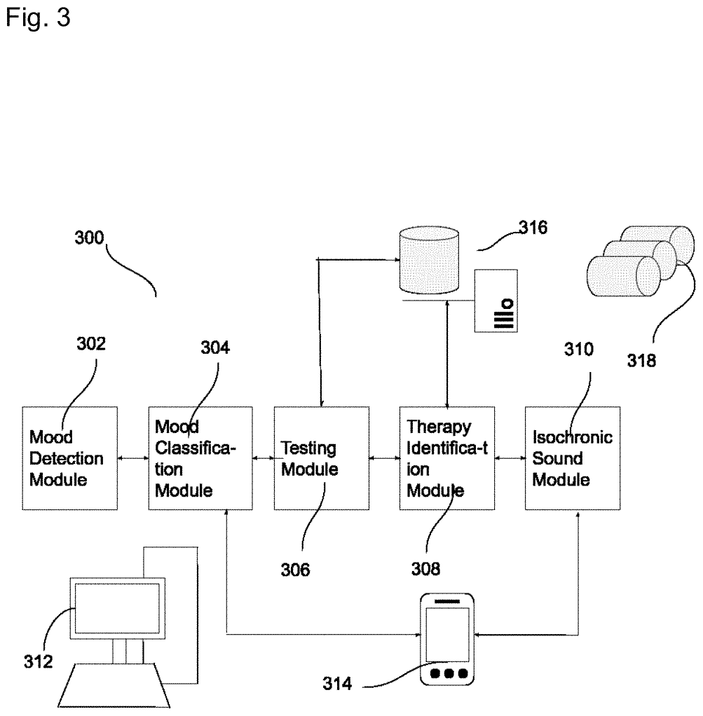

FIG. 3 illustrates an example flow diagram of a computer-based system implementing multiple modules directed to automatic client mental health state detection and assessment, classification, testing and remote isochronic sound treatment delivery in accordance with an embodiment of the present invention. A computer-based client mental health treatment system 300, which may be installed on a portable electronic peripheral device 314 such as a smartphone or tablet, as well as a traditional desktop or laptop computer, may comprise a mood detection (and assessment) module 302, a mood classification module 304, a testing module 306, a therapy identification module 308, and an isochronic sound module 310. The mood detection module 302 may have a means of detection and assessment of a mood, mental state, or condition of the client 106, e.g., as shown and discussed in FIGS. 1 and 2. In an example embodiment, the mood detection module 302 may electronically communicate with a computer 312 regarding data entry, input and/or display. Such means may include, but are not limited to, electric and/or electronic medical-related biometric equipment directed to automated sensing of a health and/or mental condition of the client 106 to provide automatic feedback regarding the same to, for example (and without limitation), a processing unit, a computer, or a server 316 to execute computer code and/or algorithms from a non-transitory computer readable medium for automatically and remotely administering client mental health treatment or therapy. In an embodiment, the client 106 can input directly, e.g., via touch-screen sensitivity of a mobile electronic peripheral such as a smartphone or tablet equipped with the mental health treatment system 300, data or information related to his or her mental health. One skilled in the art will appreciate these methods of data or information entry and input are provided as examples and that other suitable methods of data or information entry may exist without departing from the scope and spirit of the disclosed embodiments. Mood classification module 304 receives the input detected mood from the mood detection module 302 to classify the same into an appropriate category, e.g., anxiety, anger, stress, etc., and also communicates electronically with the device 314. A testing module 306 provides one or more tests or evaluations to the client 106 to determine the extent or severity of the of the mental health state, mood, or disorder, etc., and calculates a particularized and corresponding treatment plan in a therapy identification module 308. Both the testing module 306 and the therapy identification module 308 are electronically connected or coupled with a server 316 and a computer-based storage medium 318 to receive, store, and provide test data and related calculative results from a non-transitory computer readable medium. The therapy identification module 308 suggests appropriate corrective therapy commensurate with the results of the testing module 306, which are administered via an isochronic sound module 310.

As generally understood and defined herein, isochronic sounds, tones and/or waves are regular beats of a single tone used alongside monaural and binaural beats in a process referred to as “brainwave entrainment”. At its most basic level, an isochronic tone is a tone that is turned on and off rapidly creating a type of auditory pulse to result in sharp, distinctive pulses of sound. Isochronic tones can be a fast and effective audio-based way to stimulate the brain of a listener. Among several notable benefits, isochronic sounds can help improve mental focus, relaxation, energy levels, sleep and more without the need for supplemental drug-based medication or additional specialized equipment. Isochronic tones function by guiding dominant brainwave activity to a different frequency while the listener hears them, allowing for a resultant change in the mental state and feeling of the listener.

During typical operation, the client 106 inputs mood or other mental state information into the device 314 via automatic means including biometric sensors and/or touch-based input. The mood classification module 304 then processes this input mood-related data from the client 106 to classify the mood of the client into one or more categories, e.g., anxiety, stress, anger, etc. The testing module then provides one or more tests to the client 106 to determine the extent and severity of the mood state or condition of the client 106. The outcome of the tests provided by the testing module 106 are used to calculate particularized therapies corresponding to the type of mood state of the client 106, which are then administered via the isochronic sound module 310, e.g., such that the isochronic sounds 310 create a corrective and relaxing state of mind for the client 106 to address his or her mental health needs.

It may be appreciated by a person of ordinary skill in the art, in view of and in accordance with the teachings of the disclosed embodiments, that one or more modules of the mental health treatment system 300 may be embodied at least partially in a single device. In an example embodiment, all modules except for the isochronic sound module 310 may embodied in a single device, such as the device 314. In such a configuration, the isochronoic sound module 310 may be associated with a wireless speaker, such as that with Bluetooth connectivity capabilities, equipped to play isochronic sounds upon receipt of corresponding commands from the therapy identification module 308 of the mental health treatment system 300. Alternatively, the isochronic sound module 310 may be integrated with the device 314 to play isochronic sounds when directed to do so by the therapy identification module 308.

It may be appreciated by a person with ordinary skill in the art, in light of and in accordance with the teachings of the present invention, that virtually any algorithm and/or computer code may be used to detect the mood of the client in the mood detection module 302, to classify the mood of the client in the mood classification module 304, to provide tests in the testing module, to identify corresponding therapies in the therapy identification module 308, and to generate sounds in the isochronic sound module 310. Mood-related data or information detection, classification, testing, therapy identification, and isochronic sound generation algorithms may include, by way of example and without limitation, limitation, Bayesian networks, fuzzy logic, neural networks, template matching, Hidden Markov models, machine learning, data mining, feature extraction and data analysis/statistics, optical character recognition, etc.

It may be appreciated by a person with ordinary skill in the art, in light of and in accordance with the teachings of the present invention, that there may be a plurality of the same or substantially similar modules in the mental health treatment system 300. A plurality of modules such as, without limitation, mood detection module 302, mood classification module 304, testing module 306, therapy identification module 308, and isochronic sound module 310 may be present in the mental health treatment system 300. The plurality of similar modules may work sequentially in series, concurrently in parallel or independently to facilitate the detection, assessment, classification, and treatment of a mental-health related problem or issue. In an alternative configuration of the disclosed embodiments, a plurality of modules may be connected to the mental health treatment system 300 via wired and wireless connections to access resources from different wired and wireless networks.

It may be appreciated by a person with ordinary skill in the art, in view of and in accordance with the teachings of the disclosed embodiments, that one or more modules may transmit mental health client mood-related information to a tech support server, e.g., the server 316, that is on an accessible network or over the internet.

It may be appreciated by a person with ordinary skill in the art, in view of and in accordance with the teachings of the disclosed embodiments, that any module in the mental health treatment system 300 may perform data manipulation. Data manipulation is defined as including, but not being limited to, data compression, encryption, formatting. In an example embodiment of the present invention, any module sending data may first compress the data prior to data transmission.

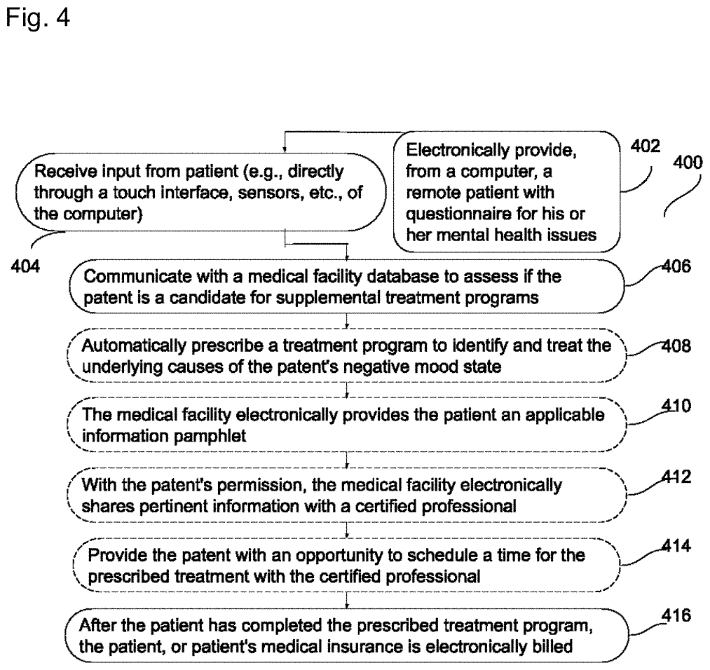

FIG. 4 illustrates an example flow diagram for electronically providing a questionnaire for client completion regarding the automated prescription of a corresponding mental health treatment method. A mental health questionnaire provision and return process 400 can, in an example embodiment, work in tandem with or otherwise supplement any one or more of the client treatment system 100 shown in FIG. 1, counseling system 200 shown in FIG. 2, and/or client mental health treatment system 300.

Prior to initiation of the mental health questionnaire provision and return process 400, mental health care related professionals can, in an example embodiment, obtain a national provider identifier (“NPI”), generally understood and defined herein to refer to a unique 10-digit identification number issue to health care providers in the United States by the Centers for Medicare and Medicaid Services (“CMS”). [Source: “HIPAA—General Information”. Centers for Medicare & Medicaid Services. Retrieved Aug. 15, 2006.] Next, a mental health care and/or medical facility, for example, can contract with the mental care professional for him or her to provide supplemental treatment programs, inclusive (but not limited to) holding classes, workshops, and other treatment programs as needed. The medical facility, e.g., psychiatric hospital, can assign a mental health care related professional and/or practitioner for each type of program envisions, e.g., specific workshops geared toward treating anxiety, depression, anger, etc. In an example embodiment, the mental health care related professional can be located in the medical facility and meet with the client therein or may be based remotely providing services via telecommuting.

After the initial preparatory relationships are established as outlined above, the mental health questionnaire provision and return process 400 begins with the electronic provision, from a computer (e.g., laptop, desktop, tablet, smartphone, smartwatch, etc.), of a questionnaire relating to the mental health concerns or issues of a client at step 402. The computer-based questionnaire provision source then receives input from the client at a step 404 directly through, for example, a touch-sensitive interface, or through biometric sensors placed on or within the client. Such client input is electronically communicated to a medical facility database, e.g., via wire or wireless telecommunications means, at a step 406 to assess if the client is a candidate for supplemental treatment programs. Should the client be identified by the medical facility database as a suitable candidate to receive supplemental treatment programs, the mental health questionnaire provision and return process 400 automatically prescribes a treatment program to identify and treat the underlying causes of the negative mood state of the client at step 408.

Next, steps 410-414 are optional and may be omitted in some embodiments. The medical facility, at step 410, can electronically provide the client an applicable information pamphlet. With the client's permission, the medical facility electronically shares pertinent information with a certified professional at step 414. The client is provided with an opportunity to schedule a time for the prescribed treatment with the certified professional at step 414. After the client has completed the prescribed treatment program, the client, or client's medical insurance, is electronically billed at step 416.

It may be appreciated by a person with ordinary skill in the art, in view and in accordance with the teaching of the disclosed embodiments, that virtually any additional steps may be added to the mental health questionnaire provision and return process 400 without departing from the scope and spirit of the disclosed embodiments. Additional steps may include, but are not limited to, additional data gathering, sending information to additional electronic devices having the mental health questionnaire provision and return process 400 installed therewith, and accessing additional resources. In an embodiment, additional medical facilities may be consulted for second opinions regarding mental health treatment methodology. In an embodiment, multiple mental health clients may be treated simultaneously by the same mental health questionnaire provision and return process 400 or multiple independent or inter-related variants thereof.

It may be appreciated by a person with ordinary skill in the art, in view and in accordance with the teachings of the disclosed embodiments, that one or more steps in the mental health questionnaire provision and return process 400 may be added, removed, or rearranged. In an embodiment, the mental health questionnaire provision and return process 400 can omit steps 410-414. In an embodiment, the order of the steps in the mental health questionnaire provision and return process 400 may occur in any order.

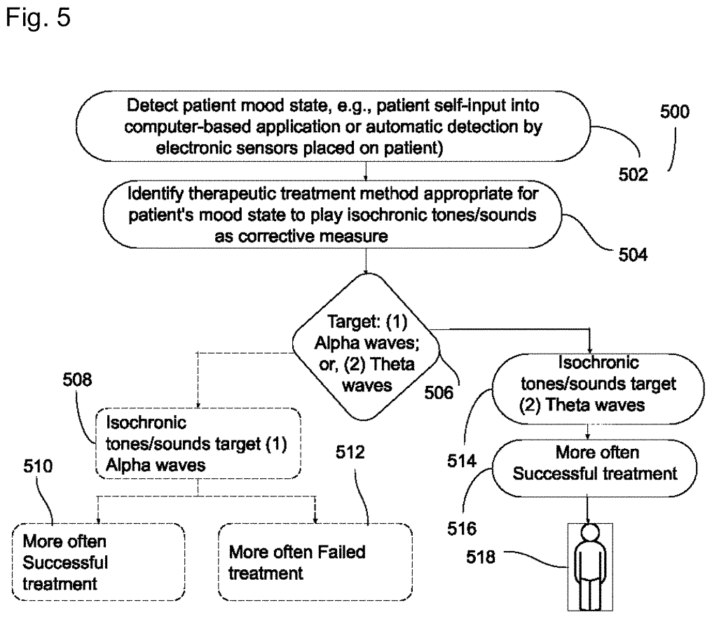

FIG. 5 illustrates an example flow diagram of a computer-based method for automated client mood detection for related treatment, the method using isochronic sounds or tones to target particular brain waves. A computer-based isochronic sound mental health treatment system 500 may, in an embodiment, work with or otherwise supplement the isochronic sound module 310 of the computer-based client mental health treatment system 300 shown in FIG. 3. The isochronic sound mental health treatment system 500 begins at step 502 with the detection of a client mood state, e.g., that may be input by the client into a computer-based application or automatic detection by electronic sensors placed on the client. Next, at step 504, a therapeutic treatment method is identified that is appropriate for the mood state of the client to initiate the playing of isochronic tones or sounds as a mood-correcting measure.

As generally understood in the art and as defined herein, isochronic tones are regular beats of a single tone that are used alongside monaural beats and binaural beats in a process named brainwave entrainment (e.g., also referred to as brainwave synchronization [Source: Fredricks, R. (2008). Healing and Wholeness: Complementary and Alternative Therapies for Mental Health. All Things Well Publications/AuthorHouse. p. 120. ISBN 978-1-4343-8336-5. Retrieved Apr. 5, 2017] and neural entrainment, referring to the hypothesized capacity of the brain to naturally synchronize its brainwave frequencies with the rhythm of periodic external stimuli, most commonly auditory, visual, or tactile.) It is widely accepted that patterns of neural brain cell activation (e.g., referred to as “firing), measured in Hz, correspond with states of alertness such as focused attention, deep sleep, etc. [Source: Cantor, David S.; Evans, James R. (2013-10-18). Clinical Neurotherapy: Application of Techniques for Treatment. Academic Press.] It is hypothesized that by listening to these beats of certain frequencies one can induce a desired state of consciousness that corresponds with specific neural activity.

Neural oscillations are rhythmic or repetitive electrochemical activity in the brain and central nervous system. Such oscillations can be characterized by their frequency, amplitude and phase. Neural tissue can generate oscillatory activity driven by mechanisms within individual neurons, as well as by interactions between them. They may also adjust frequency to synchronize with the periodic vibration of external acoustic or visual stimuli. [Source: Niedermeyer E. and da Silva F. L., Electroencephalography: Basic Principles, Clinical Applications, and Related Fields. Lippincott Williams & Wilkins, 2004.]

The activity of neurons generates electric currents; and the synchronous action of neural ensembles in the cerebral cortex, comprising large numbers of neurons, produce macroscopic oscillations. These phenomena can be monitored and graphically documented by an electroencephalogram (“EEG”). The electroencephalographic representations of those oscillations are typically denoted by the term ‘brainwaves’ in common parlance. [Source: da Silva F L (1991). “Neural mechanisms underlying brain waves: from neural membranes to networks”. Electroencephalography and Clinical Neurophysiology. 79 (2): 81-93.; Cooper R, Winter A, Crow H, Walter W G (1965). “Comparison of subcortical, cortical, and scalp activity using chronically indwelling electrodes in man”. Electroencephalography and Clinical Neurophysiology. 18 (3)].

The meaning and origin of the term “entrainment” is generally understood and defined herein as being a term originally derived from complex systems theory, and denotes the way that two or more independent, autonomous oscillators with differing rhythms or frequencies, when situated in a context and at a proximity where they can interact for long enough, influence each other mutually, to a degree dependent on coupling force, such that they adjust until both oscillate with the same frequency. Examples include the mechanical entrainment or cyclic synchronization of two electric clothes dryers placed in close proximity, and the biological entrainment evident in the synchronized illumination of fireflies. [Source: Neda Z, Ravasz E, Brechet Y, Vicsek T, Barabsi A L (2000). “Self-organizing process: The sound of many hands clapping”. Nature. 403 (6772): 849-850.].

Subsequently, the term ‘entrainment’ has been used to describe a shared tendency of many physical and biological systems to synchronize their periodicity and rhythm through interaction. This tendency has been identified as specifically pertinent to the study of sound and music generally, and acoustic rhythms specifically. The most common and familiar examples of neuromotor entrainment to acoustic stimuli is observable in spontaneous foot or finger tapping to the rhythmic beat of a song.

Brainwaves, or neural oscillations, share the fundamental constituents with acoustic and optical waves, including frequency, amplitude and periodicity. Consequently, this discovery precipitated inquiry into whether or not the synchronous electrical activity of cortical neural ensembles might not only alter in response to external acoustic or optical stimuli but also entrain or synchronize their frequency to that of a specific stimulus, e.g. sounds, tones, or other forms of auditory stimulation. [Source: Will, U., and Berg, E., “Brainwave synchronization and entrainment to periodic stimuli” Neuroscience Letters, Vol. 424, 2007, pp 55-60; Cade, G. M. and Coxhead, F., The awakened mind, biofeedback and the development of higher states of awareness. New York, N.Y.: Delacorte Press, 1979; Cade, G. M. and Coxhead, F., The awakened mind, biofeedback and the development of higher states ofawareness. New York, N.Y.: Delacorte Press, 1979; Neher, A., “Auditory driving observed with scalp electrodes in normal subjects. Electroencephalography and Clinical Neurophysiology, Vol. 13, 1961, pp 449-451; Zakharova, N. N., and Avdeev, V. M., “Functional changes in the central nervous system during music perception. Zhumal vysshei nervnoi deiatelnosti imeni IP Pavlova Vol. 32, No. 5, 1981, pp 915-924].

Thus, in view of at least the above as established by accepted empirical study, isochronic tones can be used in many different ways to impart multiple benefits upon listeners. While isochronic tones remain popular for meditation, they are increasingly being used in study to improve focus and concentration. [Source: https://www.mindamend.com/brainwave-entrainment/isochronic-tones/; Retrieved on Jun. 2, 2019]. As contemplated by the isochronic sound mental health treatment system 500, generation of particular isochronic sounds and tones corresponding to specific negative mood states changes the dominant brainwave frequency and guides the brain of the listener to a preferred to optimum state.

Human brainwave frequency is characterized into the following: (1) delta waves; (2) theta waves; (3) alpha waves; (4) beta waves; and, (5) gamma waves.

Delta waves, ranging from 0.5 to 4 Hz, are very slow and low in frequency, and are produced during the deepest stages of sleep. The human body performs most of its healing and regenerating of cells during delta wave state sleep. [Source: https://www.mindamend.com/brainwave-entrainment/isochronic-tones/; Retrieved on Jun. 2, 2019].

Theta waves, ranging from 4 Hz to 8 Hz, are also mainly dominant during sleep or in a state of deep relaxation when the brain is drifting in and out of sleep. [Source: https://www.mindamend.com/brainwave-entrainment/isochronic-tonesi; Retrieved on Jun. 2, 2019]. Research has shown that enhanced theta brain wave activity is observed in EEG data gathered from active meditation practitioners, indicating the beneficial and relaxing effects of theta brain wave targeting and manipulation. [Source: “An Electroencephalographic Study on the Zen Meditation (Zazen)”; Kasamatsu, A.; Hirai, T.; (1966) Folia Psychiatrica et Neurologica Japonica, 20, 315-226].

Alpha waves, ranging from 8 Hz to 12 Hz, are associated with a very relaxed, calm and resting awake mental state. Increases in alpha brain wave activity can result in the reduction of stress and anxiety. Alpha waves can also stimulate creativity, visualize and improve upon the brain's ability to absorb and commit information to memory. Research has shown that photic (light) stimulation can induce alpha wave activity. [Source: “The Berger Rhythm: Potential Changes from the Occipital Lobes in Man”; Adrian. E.; Matthews, B., Brain, 57, 355-384]. Stimulation of alpha brain waves has been associated with: (1) pain relief [Source: “Slow-Wave Photic Simulation Relieves Client Discomfort during Esophagogastroduodenoscopy”; Nomura, T., Higuchi, K., Yu, H., et. al.; 2006; 21 (1 Pt 1): 54-58)); (2) stress and anxiety reduction [Source: “Stress Reduction by Technology? An Experimental Study into the effects of Brainmachines on Burnout and State Anxiety”; Appl. Psychophysiol. Biofeedback, 2000; (25)2:93-101]; (3) memory improvement [Source: “10 Hz Flicker Improved Recognition Memory in Older People”; Williams, J.; Ramaswamy, D. and Oulhej, A.; 2006; BMC Neurosci., 7, 21]; (4) enhance (more powerful) meditative states [Source: “Human Anterior and Frontal Midline Theta and Lower Alpha Reflect Emotionally Positive State and Internalized Attention: High-Resolution EEG investigation of Meditation”; Aftanas, L. I.; Golocheikini, S. A.; 2001; Neuroscience Letters, 310, 57-60.]

Beta waves, ranging from 12 Hz to 40 Hz, are faster and more dominant when during wakeful consciousness during the day when persons are focused, energized and alert.

Gamma waves, ranging above 40 Hz, are the fastest in the brainwave range and have been connected to mental states of high focus, cognitive enhancement, and information processing.

The human brain, when feeling strong negative emotions of stress, agitation, anxiety, or anger, produces increased amounts of beta wave activity. Therefore, application of low beta or alpha frequency isochronic tones will lower the dominant frequency, reducing such feelings of stress and or anxiety. [Source: https://www.mindamend.com/brainwave-entrainment/isochronic-tones/; Retrieved on: Jun. 2, 2019].

For the purposes of meditation and relaxation, it can be harder for new practitioners to reach deeper meditative states. Using isochronic sounds targeting low alpha and theta frequencies helps guiding the brain to these deeply relaxed mental states. [Source: https://www.mindamend.com/brainwave-entrainment/isochronic-tonesi; Retrieved on: Jun. 2, 2019]

Returning to the isochronic sound mental health treatment system 500 shown in FIG. 5, either alpha brain waves or theta brain waves may be targeted at step 506 by the isochronic sounds generated in response to identification of therapeutic treatment methods appropriate to treat the mental mood or state of a client. Historically, many tests and studies have targeted alpha waves (given their prevalence during normal wakefulness), and may be targeted at step 508 to produce either a successful treatment outcome at step 510 or a failed treatment outcome at step 512.

However, and unexpectedly in view of the conventional wisdom in the art, targeting theta brain waves at step 514 with isochronic sounds or tones yields particular beneficial results within test group populations. By way of example, and not limitation, Applicant conducted an experiment where a group of 150 test subjects were treated with isochronic sounds targeting alpha brain wave activity, and it was observed that treatment failed approximately 80% of the time. In contrast, in a group of 30 test subjects treated with isochronic sounds targeting theta brain wave activity, treatment failed only approximately 10-20% of the time, representing a significant improvement in therapeutic efficacy over isochronic sound treatment targeting alpha brain waves. Therefore, isochronic tones generated to target theta brain wave activity at step 514 is expected to be more likely to result in a successful treatment outcome at step 516 of a client 518 facing mental health related issues.

The isochronic sounds emitted at step 514 may be output by a conventional loudspeaker integrated or otherwise associated with a portable electronic peripheral having the computer-based isochronic sound mental health treatment system 500 installed therein. In an example embodiment, the client 518 may activate the isochronic tones targeting theta brain waves at step 514 by pressing a corresponding touch-sensitive area on the electronic peripheral when prompted to do so. Alternatively, in an example embodiment, the electronic peripheral may be equipped with Bluetooth® or similar wireless connectivity to electronically instruct a speaker wirelessly tethered to the electronic peripheral to initiate playing of the isochronic sounds at step 514.

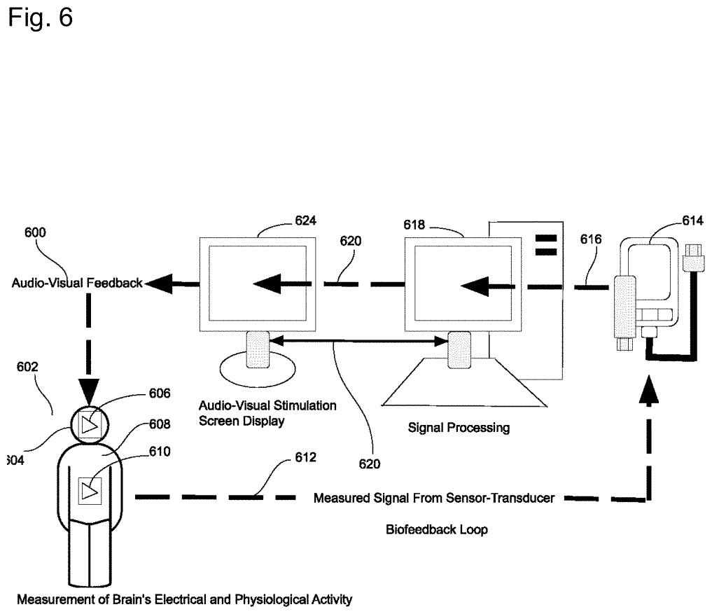

FIG. 6 illustrates an example biofeedback loop 600 for sensor and computer-based measurement of the brain's electrical and physiological activity. In an embodiment, the biofeedback loop 600 may electronically communicate or otherwise couple with any one or more of the client treatment system 100, counseling system 200, computer-based client mental health treatment system 300, mental health questionnaire provision and return process 400, and computer-based isochronic sound mental health treatment system 500 shown in FIGS. 1-5, respectively, to provide client mental health related information as needed, e.g., accommodating circumstances and configurations where the client does not himself or herself manually enter mood-related data via, for example, a touch-screen interface of a smart phone or device having any one or more of the systems and processes 100-600 installed therein.

The biofeedback loop 600 seeks to measure the brain's electrical and physiological activity via the placement of one or more biometric sensors 602, 610 that may be placed on a head 604 or a body 608 of a client 502, respectively. As conventionally understood in the art, a biometric sensor is a transducer that changes a biometric trait of a person into an electrical signal. Biometric traits mainly include biometric fingerprint reader, iris, face, voice, etc. [Source: https://www.elprocus.com/different-types-biometric-sensors/; Retrieved: Jun. 2, 2019]. In an embodiment, other types of sensors, probes or medical devices that penetrate into the skin or organs and/or brain of the client 602 may be used to obtain specific data or information not otherwise attainable from traditional on-the-skin or surface of the body biometric sensors. Such biometric data and information obtained by the biometric sensors 602, 610 is electronically communicated along a pathway 612 to a centralized computing hub 614 that further relays the biometric data or information to a signal processing workstation 618 for subsequent digital processing if desired. Next, the processed biometric data and information is electronically communicated along a pathway 620, indicative of electronic connection, between the signal processing workstation 618 and an audio-visual stimulation screen display 624 allowing for interested parties such as medical and/or mental health care professional to observe biometric feedback to diagnose and treat mental health related symptoms. One skilled in the art that various other configurations and orientations of the biofeedback loop 600 may exist including additional or fewer pieces of computing and/or network equipment without departing from the scope and spirit of the disclosed embodiments. Further, not all components shown in the biofeedback loop 600 may be necessary for successful operation, and not all components need to be situated in the same geographical location for successful operation. That is, the client 602 may be remotely located, e.g., at home, and communicate the biometric data electronically to a psychiatrist and mental health care worker, both located at different work sites away from the client's home, etc.

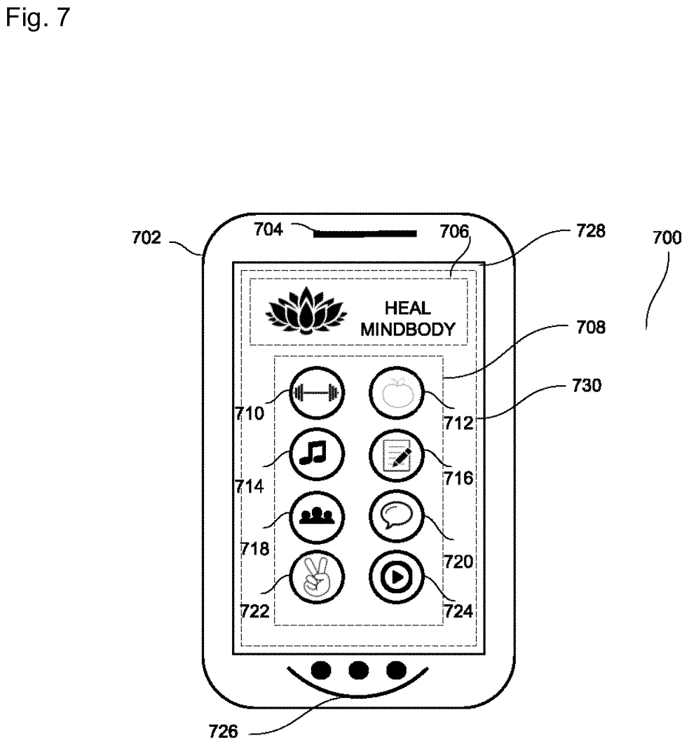

FIG. 7 illustrates an example guided user interface (“GUI”) digital medium environment 700 implemented in a portable electronic peripheral device for automatic client mental health state detection, assessment, classification, testing and remote treatment delivery. A user interface device 702 comprises of a display 728, which, in an embodiment, is touch-sensitive and thus also provides for a user input means. The display 702 may be virtually any means of delivering information to a user such as, but not limited to, a touchscreen display, a speaker, e-paper. The display 728 may also include a graphical user interface 730 which may allow a user to navigate through steps in an automatic remote mental health detection, assessment and treatment process, such as any one or more of the systems and processes 100-600, and/or perform additional functions. The graphical user interface 730 may be virtually any kind of interface that a user may interact with via a user input means, which may be virtually any hardware capable of receiving user input such as, but not limited to, a touchscreen, a keypad, a mouse, a keyboard, etc.

It may be appreciated by a person with ordinary skill in the art, in view of and in accordance with the teachings of the present invention, that one or more elements of user interface device 702 may be comprised of one or more individual devices. In another embodiment of the present invention, all the elements of user interface device 702 may be embodied in a single smartphone device. In still another embodiment of the present invention, a user input means may be a separate hardware device from the display 730.

It may be appreciated by a person with ordinary skill in the art, in light of and in accordance with the teachings of the present invention, that graphical user interface 730 may be formatted based on user interface device 702. Graphical user interface 730 may be formatted to meet a specific user interface device 702's hardware specifications such as, without limitation, a display resolution, a processing speed, an amount of available RAM, etc. In another embodiment of the present invention, graphical user interface 730 may be virtually any resolution to accommodate a user interface device 702's display specifications.

In an embodiment, the graphical user interface 730 may include any one or more of individual icons 710-724, each icon corresponding to distinct functionality related to mental health client mood detection and therapy. For instance, an exercise icon 710 may refer to the importance of fitness to overall health and happiness of the client, allowing for the transformation of the body with enumerated fitness routines. A nutrition icon 712 may refer to keeping track of a detailed calorie log and other tabulated nutritional information. A meditation icon 714 can provide meditative guidance allowing a mental health patience to experience mindfulness and inner well-being. A task-management icon 716 can coordinate and organize the completion of specific tasks. A groups 718 icon can allow for the communication between multiple mental health clients and/or mental health care professionals in a structured and organized setting geared towards healing and mutual encouragement. A chat icon 720 can provide for the contact means between specific mental health clients and their respective mental health care providers, as well as for the sharing of inspirational stories and successes. A challenges icon 722 can track personal growth and accepting defined goals and setting new ones. And, a video icon 724 can provide for educational content geared to focus the mind of the mental health client. One skilled in the art will appreciate that the icon list 710-724 is provided as an example and is thus not exhaustive and that additional or fewer icons representative of mental health detection and treatment functionality may exist without departing from the scope and spirit of the disclosed embodiments, as may exist alternative configurations or placements of the icons 710-724 on or within the graphical user interface 730.

One skilled in the art will appreciate that the general configuration of the icons 710-724 within the graphical user interface 730 impart specific computer-based functionality integrally tied into the provision of isochronic sound waves uniquely targeting theta brain wave frequencies. The real-time interaction provided by each icon 710-724 triggers calculative feedback resulting in the optimization and deliverance of appropriate isochronic sound waves, e.g., via the isochronic sound module 310 of the computer-based client mental health treatment system 300 shown in FIG. 3, targeting theta brain wave frequencies specific to individual negative mood states, e.g., anxiety, stress, etc.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

Such computers referenced and/or described in this disclosure may be any kind of computer, either general purpose; or some specific purpose computer such as, but not limited to, a workstation, a mainframe, GPU, ASIC, etc. The programs may be written in C, or Java, Brew or any other suitable programming language. The programs may be resident on a storage medium, e.g., magnetic or optical, e.g., without limitation, the computer hard drive, a removable disk or media such as, without limitation, a memory stick or SD media, or other removable medium. The programs may also be run over a network, for example, with a server or other machine sending signals to the local machine, which allows the local machine to carry out the operations described herein.

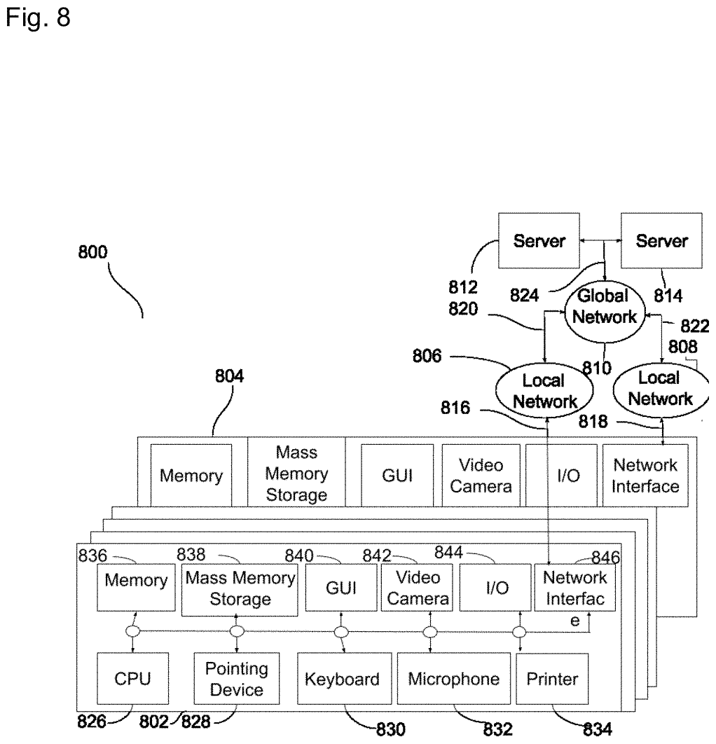

FIG. 8 is a block diagram depicting an exemplary client/server system which may be used by an example web-enabled/networked embodiment of the disclosed embodiments.

A communication system 800 includes a multiplicity of clients with a sampling of clients denoted as a client 802 and a client 804, a multiplicity of local networks with a sampling of networks denoted as a local network 806 and a local network 808, a global network 810 and a multiplicity of servers with a sampling of servers denoted as a server 812 and a server 814.

Client 802 may communicate bi-directionally with local network 806 via a communication channel 816. Client 804 may communicate bi-directionally with local network 808 via a communication channel 818. Local network 806 may communicate bi-directionally with global network 810 via a communication channel 820. Local network 808 may communicate bi-directionally with global network 810 via a communication channel 822. Global network 810 may communicate bi-directionally with server 812 and server 814 via a communication channel 824. Server 812 and server 814 may communicate bi-directionally with each other via communication channel 824.

Furthermore, clients 802,804, local networks 806,808, global network 810 and servers 812,814 may each communicate bi-directionally with each other.

In one embodiment, global network 810 may operate as the Internet. It will be understood by those skilled in the art that communication system 800 may take many different forms. Non-limiting examples of forms for communication system 800 include local area networks (LANs), wide area networks (WANs), wired telephone networks, wireless networks, or any other network supporting data communication between respective entities.

Clients 802 and 804 may take many different forms. Non-limiting examples of clients 802 and 804 include personal computers, personal digital assistants (PDAs), cellular phones and smartphones.

Client 802 includes a CPU 826, a pointing device 828, a keyboard 830, a microphone 832, a printer 834, a memory 836, a mass memory storage 838, a GUI 840, a video camera 842, an input/output interface 844 and a network interface 846.

CPU 826, pointing device 828, keyboard 830, microphone 832, printer 834, memory 836, mass memory storage 838, GUI 840, video camera 842, input/output interface 844 and network interface 846 may communicate in a unidirectional manner or a bi-directional manner with each other via a communication channel 848.

Communication channel 848 may be configured as a single communication channel or a multiplicity of communication channels.

CPU 826 may be comprised of a single processor or multiple processors. CPU 826 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general-purpose microprocessors.

As is well known in the art, memory 836 is used typically to transfer data and instructions to CPU 826 in a bi-directional manner. Memory 836, as discussed previously, may include any suitable computer-readable media, intended for data storage, such as those described above excluding any wired or wireless transmissions unless specifically noted. Mass memory storage 838 may also be coupled bi-directionally to CPU 826 and provides additional data storage capacity and may include any of the computer-readable media described above. Mass memory storage 838 may be used to store programs, data and the like and is typically a secondary storage medium such as a hard disk. It will be appreciated that the information retained within mass memory storage 838, may, in appropriate cases, be incorporated in standard fashion as part of memory 836 as virtual memory.

CPU 826 may be coupled to GUI 840. GUI 840 enables a user to view the operation of computer operating system and software. CPU 826 may be coupled to pointing device 828. Non-limiting examples of pointing device 828 include computer mouse, trackball and touchpad. Pointing device 828 enables a user with the capability to maneuver a computer cursor about the viewing area of GUI 840 and select areas or features in the viewing area of GUI 840. CPU 826 may be coupled to keyboard 830. Keyboard 830 enables a user with the capability to input alphanumeric textual information to CPU 826. CPU 826 may be coupled to microphone 832. Microphone 832 enables audio produced by a user to be recorded, processed and communicated by CPU 826. CPU 826 may be connected to printer 834. Printer 834 enables a user with the capability to print information to a sheet of paper. CPU 826 may be connected to video camera 842. Video camera 842 enables video produced or captured by user to be recorded, processed and communicated by CPU 826.

CPU 826 may also be coupled to input/output interface 844 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 826 optionally may be coupled to network interface 846 which enables communication with an external device such as a database or a computer or telecommunications or internet network using an external connection shown generally as communication channel 816, which may be implemented as a hardwired or wireless communications link using suitable conventional technologies. With such a connection, CPU 826 might receive information from the network, or might output information to a network in the course of performing the method steps described in the teachings of the present invention.

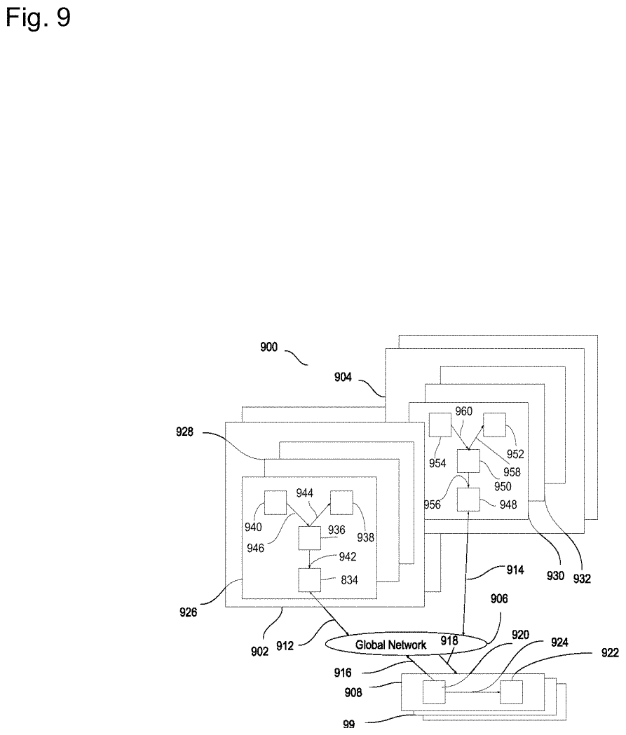

FIG. 9 illustrates a block diagram depicting an example client/server communication system that may be used for automatic remote client mental health detection, assessment, and treatment. A communication system 900 includes a multiplicity of networked regions with a sampling of regions denoted as a network region 902 and a network region 904, a global network 906 and a multiplicity of servers with a sampling of servers denoted as a server device 908 and a server device 99.

Network region 902 and network region 904 may operate to represent a network contained within a geographical area or region. Non-limiting examples of representations for the geographical areas for the networked regions may include postal zip codes, telephone area codes, states, counties, cities and countries. Elements within network region 902 and 904 may operate to communicate with external elements within other networked regions or within elements contained within the same network region.

In some implementations, global network 906 may operate as the Internet. It will be understood by those skilled in the art that communication system 900 may take many different forms. Non-limiting examples of forms for communication system 900 include local area networks (LANs), wide area networks (WANs), wired telephone networks, cellular telephone networks or any other network supporting data communication between respective entities via hardwired or wireless communication networks. Global network 906 may operate to transfer information between the various networked elements.

Server device 908 and server device 99 may operate to execute software instructions, store information, support database operations and communicate with other networked elements. Non-limiting examples of software and scripting languages which may be executed on server device 908 and server device 99 include C, C++, C# and Java.

Network region 902 may operate to communicate bi-directionally with global network 906 via a communication channel 912. Network region 904 may operate to communicate bi-directionally with global network 906 via a communication channel 914. Server device 908 may operate to communicate bi-directionally with global network 906 via a communication channel 916. Server device 99 may operate to communicate bi-directionally with global network 906 via a communication channel 918. Network region 902 and 904, global network 906 and server devices 908 and 99 may operate to communicate with each other and with every other networked device located within communication system 900.

Server device 908 includes a networking device 920 and a server 922.

Networking device 920 may operate to communicate bi-directionally with global network 906 via communication channel 916 and with server 922 via a communication channel 924. Server 922 may operate to execute software instructions and store information.

Network region 902 includes a multiplicity of clients with a sampling denoted as a client 926 and a client 928. Client 926 includes a networking device 934, a processor 936, a GUI 938 and an interface device 940. Non-limiting examples of devices for GUI 938 include monitors, televisions, cellular telephones, smartphones and PDAs (Personal Digital Assistants). Non-limiting examples of interface device 940 include pointing device, mouse, trackball, scanner and printer. Networking device 934 may communicate bi-directionally with global network 906 via communication channel 912 and with processor 936 via a communication channel 942. GUI 938 may receive information from processor 936 via a communication channel 944 for presentation to a user for viewing. Interface device 940 may operate to send control information to processor 936 and to receive information from processor 936 via a communication channel 946. Network region 904 includes a multiplicity of clients with a sampling denoted as a client 930 and a client 932. Client 930 includes a networking device 948, a processor 950, a GUI 952 and an interface device 954. Non-limiting examples of devices for GUI 938 include monitors, televisions, cellular telephones, smartphones and PDAs (Personal Digital Assistants). Non-limiting examples of interface device 940 include pointing devices, mousse, trackballs, scanners and printers. Networking device 948 may communicate bi-directionally with global network 906 via communication channel 914 and with processor 950 via a communication channel 956. GUI 952 may receive information from processor 950 via a communication channel 958 for presentation to a user for viewing. Interface device 954 may operate to send control information to processor 950 and to receive information from processor 950 via a communication channel 960.

For example, consider the case where a user interfacing with client 926 may want to execute a networked application. A user may enter the IP (Internet Protocol) address for the networked application using interface device 940. The IP address information may be communicated to processor 936 via communication channel 946. Processor 936 may then communicate the IP address information to networking device 934 via communication channel 942. Networking device 934 may then communicate the IP address information to global network 906 via communication channel 912. Global network 906 may then communicate the IP address information to networking device 920 of server device 908 via communication channel 916. Networking device 920 may then communicate the IP address information to server 922 via communication channel 924.

Server 922 may receive the IP address information and after processing the IP address information may communicate return information to networking device 920 via communication channel 924. Networking device 920 may communicate the return information to global network 906 via communication channel 916. Global network 906 may communicate the return information to networking device 934 via communication channel 912. Networking device 934 may communicate the return information to processor 936 via communication channel 942. Processor 946 may communicate the return information to GUI 938 via communication channel 944. User may then view the return information on GUI 938.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC § 112 (6)/(f) it is intended that the corresponding means for and/or steps for carrying out the claimed function are the ones that are locally implemented within the jurisdiction of the USA, while the remaining aspect(s) performed or located remotely outside the USA are not intended to be construed under 35 USC § 112 (6) pre-AIA or 35 USC § 112 (f) post AIA. In some embodiments, the methods and/or system components which may be located and/or performed remotely include, without limitation: mental health client data or information entry, e.g., either via manual self-entry by a touch-sensitive screen of a portable electronic peripheral (“smartphone”) and/or through biometric sensing and/or feedback; hospital receipt of mental client data for consideration and evaluation to interact with inventive processes and methods to suggest and/or prescribe a therapeutic method, etc.

It is noted that according to USA law, all claims must be set forth as a coherent, cooperating set of limitations that work in functional combination to achieve a useful result as a whole. Accordingly, for any claim having functional limitations interpreted under 35 USC § 112 (6)/(f) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC § 112 (6)/(f) is/(are) dependent on one or more remote servers located outside the USA, it is intended that each such recited function under 35 USC § 112 (6)/(f) is to be interpreted as the function of the local system receiving the remotely generated information required by a locally implemented claim limitation, wherein the structures and or steps which enable, and breathe life into the expression of such functions claimed under 35 USC § 112 (6)/(f) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present patent application, and “35 USC § 112 (6)/(t)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(t)) functional limitation would have to be conducted to support a legally valid Examination on that USPTO policy for broadest interpretation of “mean for” claims. Accordingly, the USPTO wiUhave discovered a multiplicity of prior art documents including disclosure of specific structures and elements which are suitable to act as corresponding structures to satisfy all functional limitations in the below claims that are interpreted under 35 USC § 112(6) (post AIA 112 (f)) when such corresponding structures are not explicitly disclosed in the forgoing patent specification. Therefore, for any invention element(s)/structure(s) corresponding to functional claim limitation(s), in the below claims interpreted under 35 USC § 112(6) (post AIA I 12(t)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA I 12(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AJA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing for automatic client mental health state detection, assessment, classification, testing and remote treatment delivery according to the present invention will be apparent to those skilled in the art. Various aspects of the invention have been described above by way of illustration, and the specific embodiments disclosed are not intended to limit the invention to the particular forms disclosed. The particular implementation of the automatic client mental health state detection, assessment, classification, testing and remote treatment delivery may vary depending upon the particular context or application. By way of example, and not limitation, the automatic client mental health state detection, assessment, classification, testing and remote treatment delivery described in the foregoing were principally directed to automatic detection implementations for mental health clients located remote from their corresponding medical and/or mental care facilities; however, similar techniques may instead be applied to circumstances and/or configurations in which the mental health client and concerned care providers are located in the same general area and/or any combination of localized and/or remote geographical positionings, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section I.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre AIA) or 35 USC 112(t) post-AIA. Otherwise, no limitations from the specification are to be read into any claims, unless those limitations are expressly included in the claims.

Although the present disclosure has been explained in relation to its preferred embodiment(s) as mentioned above, it is to be understood that many other possible modifications and variations can be made without departing from the spirit and scope of the inventive aspects of the present invention. It is, therefore, contemplated that the appended claim or claims will cover such modifications and variations that fall within the true scope of the invention.

What we claim is:

1. A system to help businesses and clinics provide mental health care services 24 hours anywhere and provide mental health assessment and mental health illness treatment to clients to address the root causes and triggers of mental health illness and negative emotional states and conditions.

2. The system of claim 1 comprising a website and mobile application to make the mental health care services available 24 hours all the day where a client can receive the mental health care services provided even when a time is not in the working hours of the business or clinic.

3. The system of claim 1 comprising a website and mobile application to make the mental health care services available anywhere where a client can receive the health care services even if a client is not in the place of business.
The system of claim 1 is to help companies provide mental health care support, career development and self development support to improve the employee career development, improve employees performance, efficiency and productivity by addressing the root causes and triggers of mental health illness and negative emotional states and conditions, and low job performance through a system comprising the steps of:
The system of claim . . . comprising a website and mobile application to make the mental health care support, career development and self development support provided to employees available 24 hours all the day where an employee can receive the mental health care support, career development and self development support even when a time is not in the working hours of the company.
The system of claim . . . comprising a website and mobile application to make the mental health care support, career development and self development support provided to employees available anywhere where an employee can receive the mental health care support, career development and self development support even when the employee does not exist in the company.
The system of claim 1 is to help people get self development support 24 hours everywhere.

4. A method to help businesses and clinics provide mental health care services, mental health assessment and mental health diseases treatment 24 hours anywhere to clients to address the root causes and triggers of mental health illness and negative emotional states and conditions through a system comprising the steps of:
a business or clinic signs up for an account on the system;
a business or clinic signs up its clients to the system upon visitation and encourages clients to access the system;
a business or clinic customizes the mental health care services and treatments provided through the system to fit clients' needs; and,
a client logs in to the system to have a resource of treatment, accessible anytime when feeling any of the symptoms of mental illness.

5. The method as claimed in claim 4 wherein a client accesses their account on the system to choose mental health care services comprising:
stress and anxiety tests;
guided Meditations;
video therapies;
exercise routines;
nutrition log;
task management;
groups;
challenges; and,
chat.

6. The stress and anxiety tests as claimed in claim 5 wherein a client will answer a series of questions to determine how a client is feeling, whether a client depressed, overwhelmed, sad, stressed or anxious, each question comprising four weighted responses, the responses totals are tallied after the line of questioning is complete to determine the severity of the client's mental state, and recommendations are generated for treatments that could help mental health patent relieve some of these negative feelings, these recommendations comprising relevant contact information of business or clinics, advice, guided meditations and video therapies.

7. The Guided Meditations as claimed in claim 5 wherein the system integrates subliminal isochronic tones to aid for treatment of mental health illness and achieve results specified by the business or clinic, where there are guided meditations designed to treat a variety of client cases, where the guided meditations treat emotional states of a client may be going through stress relief, depression, confidence, motivation or various fears.

8. The video therapies as claimed in claim 5 wherein the system provide accessible therapies and treatments through videos to clients at any time to enable clients to continue their healing any time of the day.

9. The exercise routines as claimed in claim 5 wherein the system provide exercise accessible to clients at any time to enable clients to continue their healing any time of the day.

10. The nutrition Log as claimed in claim 5 wherein the system provide nutrition information accessible to clients at any time to enable clients to eat healthy.

11. The task Management as claimed in claim 5 wherein the system comprising task management software which helps clients address overwhelming mental states and find stress relief outside the place of business or clinic.

12. The task management as claimed in claims 5 and 11 including a method which helps clients address overwhelming mental states and find stress relief outside the place of business or clinic comprising the steps of:
the mental health services provider will customize the task management software by programme the software with the appropriate information;
the task management software explaining to the client how the mind processes and stores information, and how thoughts when compiled can create stress; and,
the task management software explaining to the client how to organize these thoughts by having them write down the first five tasks that come to mind, prioritizing them, and allowing them to mark them off one by one as completed, clients are encouraged to remain concise and actionable by limiting the input character count of each task.

13. The groups as claimed in claim 5 wherein clients can build communities of users with similar goals, interests, and challenges to help clients encourage each other and to help clients inspire themselves and other clients to stay on track with their treatment and recovery.

14. The challenges as claimed in claim 5 to let clients challenge each other to inspire themselves to stay on track with their treatment and recovery.

15. The chat as claimed in claim 5 is to help clients reach out to the services providers of mental health care at any time with questions or for advice.

16. The system of claim 1 and the method as claimed in claim 4 wherein the business or clinic can treat the root cause to the mental health illness wherein the root cause is almost always related to feelings, emotions, and belief systems.

17. The system of claim 1 and the method as claimed in claim 4 wherein the business or clinic can provide in house programs to provide their clients supplemental treatments to help reduce stress and thus provide better overall care.

18. The system of claim 1 and the method as claimed in claim 4 wherein a client has a resource accessible anywhere and anytime feelings of stress, anxiety, depression, or overwhelmed to help him feel good.

19. The system of claim 1 and the method as claimed in claim 4 wherein the system provides testing of symptoms of mental illness and negative emotional states resulting in individualized therapeutic solutions.

20. The system of claim 1 and the method as claimed in claim 4 wherein the business or clinic can enable a client to take what they learn in a clinical environment and practice it on a daily basis, and when negative emotional states arise, those that may trigger unwanted outcomes such as relapse, clients can address the root cause of it immediately through mobile therapy access.

21. A system to help companies provide mental health care support, career development and self development support 24 hours to improve the employee career development, improve employees performance, efficiency and productivity by addressing the root causes and triggers of mental health illness and negative emotional states and conditions, and low job performance.

22. The system of claim 21 comprising a website and mobile application to make the mental health care support, career development and self development support provided to employees available 24 hours all the day where an employee can receive the mental health care support, career development and self development support even when a time is not in the working hours of the company.

23. The system of claim 1 is to help people get self development support 24 hours everywhere.

24. A method to help companies provide mental health care support, career development and self development support to improve the employee career development, improve employees performance, efficiency and productivity by addressing the root causes and triggers of mental health illness and negative emotional states and conditions, and low job performance through a system comprising the steps of:
a company signs up for an account on the system;
a company signs up its employees to the system;
a company customizes the mental health care support, career development and self development services provided through the system to fit employee' needs; and,
An employee login to the system to have a resource of mental health care, career development and self development anytime when feeling he need a support.

25. The method as claimed in claim 21 wherein an employee accesses his account on the system to choose mental health care, career development and self development services comprising:
stress and anxiety tests;
guided Meditations;
video therapies;
exercise routines;
nutrition log;
task management;
groups;
challenges;
Chat;
career development; and,
self development

26. The stress and anxiety tests as claimed in claim 22 wherein an employee will answer a series of questions to determine how the employee is feeling, whether the employee depressed, overwhelmed, sad, stressed, anxious, need a career support, or need a self development support, each question comprising four weighted responses, the responses totals are tallied after the line of questioning is complete to determine the severity of the employee's mental state, and recommendations are generated for treatments that could help employee mental health relieve negative feelings, and get guidation about home to improve his job and career performance.

27. The Guided Meditations as claimed in claim 22 wherein the system integrates subliminal isochronic tones and guided meditation to aid employee overcome any negative emotion and make the employee take some rest to achieve the results specified by the company.

28. The video therapies as claimed in claim 22 wherein the system provide accessible career guidance through videos to employee at any time to enable employees continue getting career and self development support.

29. The exercise routines as claimed in claim 22 wherein the system provide exercise accessible to employees at any time to enable employees to overcome any negative feeling.

30. The nutrition Log as claimed in claim 22 wherein the system provide nutrition information accessible to employees at any time to enable eating healthy.

31. The task Management as claimed in claim 22 wherein the system comprises task management software which helps employees addressing managing their tasks and time according to the company policy.

29. The task management as claimed in claims 22 and 28 including a method which helps employee address overwhelming mental states and find stress relief outside the place of business or clinic comprising the steps of:
the company will customize the task management software by programme the software with the appropriate information;
the task management software explaining to the employee how the mind processes and stores information, and how thoughts when compiled can create stress; and,
the task management software explaining to the employee how to organize these thoughts by having them write down the first five tasks that come to mind, prioritizing them, and allowing them to mark them off one by one as completed, clients are encouraged to remain concise and actionable by limiting the input character count of each task.

32. The groups as claimed in claim 22 wherein employee can build communities of users with other employees have similar goals, interests, and challenges to help employees encourage each other and to help clients inspire themselves and other employees to stay on track with their goals.

33. The challenges as claimed in claim 22 is to let employees challenge each other to inspire themselves to stay on track with their goals.
The chat as claimed in claim 22 is to help employee reach out to the company at any time with questions or for advice.

16. The system of claim 1 and the method as claimed in claim 4 wherein the company can treat the root cause to the mental health illness wherein the root cause is almost always related to feelings, emotions, and belief systems.

17. The system of claim 1 and the method as claimed in claim 4 wherein the company can provide in house programs to provide their clients supplemental treatments to help reduce stress and thus provide better overall care.

18. The system of claim 1 and the method as claimed in claim 4 wherein an employee has a resource accessible anywhere and anytime feelings of stress, anxiety, depression, or overwhelmed to help him feel good.

19. The system of claim 1 and the method as claimed in claim 4 wherein the system provides testing of symptoms of mental illness and negative emotional states resulting in individualized therapeutic solutions.

20. The system of claim 1 and the method as claimed in claim 4 wherein the company can enable an employee to take what they learn in the work environment and practice it on a daily basis, and when negative emotional states arise, those that may trigger unwanted outcomes such as relapse, clients can address the root cause of it immediately through mobile therapy access.
The method of claim guitar comprising a website and mobile application to make the mental health care, career development and self development services provided to employees available 24 hours all the day where an employee can receive the career development support even when a time is not in the working hours of the company.
The method of claim 21 comprising a website and mobile application to make the mental health care, career development and self development services provided to employees available anywhere where an employee can receive the career development support even when the employee does not exist in the company.
